(19) United States
(12) Patent Application Publication
(10) Pub. No.: US 2023/0338950 A1
(43) Pub. Date: Oct. 26, 2023

(54) MICROFLUIDIC ASSAY DEVICE

(71) Applicant: Duke University, Durham, NC (US)

(72) Inventors: Ashutosh Chilkoti, Durham, NC (US); David Kinnamon, Durham, NC (US); Cassio Fontes, Durham, NC (US); Angus Hucknall, Durham, NC (US); Jason Liu, Durham, NC (US); Jacob Heggestad, Durham, NC (US)

(21) Appl. No.: 18/042,032

(22) PCT Filed: Aug. 20, 2021
(86) PCT No.: PCT/US2021/046833

§ 371 Date: Feb. 17, 2023

Related U.S. Application Data
(60) Provisional application No. 63/116,511, filed on Nov. 20, 2020.
(60) Provisional application No. 63/068,432, filed on Aug. 21, 2020.

(51) Int. Cl. B01L 3/00; G01N 33/543

(52) U.S. Cl. CPC B01L 3/502715; G01N 33/54366; B01L 2200/12; B01L 2200/16; B01L 2300/0887; B01L 2300/12; B01L 2400/0406; B01L 2400/0457

(57) ABSTRACT

Described herein is an assay device, comprising a substrate comprising microfluidic geometry, and a reagent layer disposed adjacent to the substrate.

### CROSS-REFERENCE TO RELATED APPLICATIONS

This application claims priority to U.S. Provisional Pat. Application Nos. 63/068,432, filed Aug. 21, 2020, and 63/116,511, filed Nov. 20, 2020, each of which is hereby incorporated by reference herein in its entirety.

### FEDERALLY SPONSORED RESEARCH

This invention was made with government support under grant numbers UH3CA211232 and 1R01CA248491 awarded by the National Institutes of Health; Federal Grant nos. DM160400 and W81XWH-16-C-0219 awarded by the US Army Medical Research and Material Command (AMRMC); and Federal Grant no. CBET2029361awarded by the National Science Foundation. The government has certain rights in the invention.

### TECHNICAL FIELD

Described herein is an assay device, comprising a substrate comprising microfluidic geometry, and a reagent layer disposed adjacent to the substrate.

### BACKGROUND

Recent innovations in the field of specimen analysis include a self-contained immunoassay platform fabricated upon a “non-fouling” poly(oligoethylene glycol methyl ether methacrylate) (POEGMA) brush, where reagents needed to complete the assay are inkjet-printed directly onto the surface (see e.g., U.S. Pat. Application Publication No. US 2018/0217136 A1, which is incorporated by reference herein for such teachings).

Although this technology has improved the speed and accuracy of biomarker assays, there is a need to automate and expand the capabilities of these types of devices.

### SUMMARY

One embodiment described herein is a multi-layered microfluidic assay device comprising a cassette comprising a plurality of layers comprising: a substrate layer comprising a non-fouling polymer layer coated on a glass substrate; and assay reagents disposed upon the substrate layer; a microfluidic layer comprising a channel and a reaction area in fluid communication with each other and to inlets and outlets, the ultimate microfluidic layer adjacent to a cover layer. In one aspect, the microfluidic layer comprises: a channel layer comprising a continuous circuitous channel in fluid communication with a reaction layer; and a reaction layer comprising cutouts for a reaction chamber, an inlet, and an outlet, each in fluid communication with the channel layer, the reaction layer being sandwiched between the channel layer on one side and the cover layer on the other side. In another aspect, the reaction chamber comprises an upper reaction chamber, a lower reaction chamber, and an offset mixing channel fluidly connecting the upper and lower reaction chambers. In another aspect, the offset mixing channel comprises a P-trap bend to prevent clogging. In another aspect, the assay reagents comprise one or more detection reagents and one or more capture reagents that are spatially separated and disposed on the non-fouling polymer layer to align with the lower and upper reaction chambers. In another aspect, the detection reagents are placed on layers of trehalose, disposed on the substrate spatially at a position corresponding to the upper reaction chamber; and of the capture reagents are disposed on the substrate spatially at a position corresponding to the lower reaction chamber. In another aspect, the cover layer is attached to the reaction layer via an adhesive layer. In another aspect, the channel layer, the reaction layer, and the cover layer are interconnectedly vented to ambient atmospheric pressure. In another aspect, the cover layer comprises one or more inlets and outlets in fluid communication with the microfluidic layers and with a reservoir at the inlets and an absorbent waste pad at the outlets. In another aspect, the non-fouling polymer layer is a poly(oligoethylene glycol methyl ether methacrylate) (POEGMA). In another aspect, the channel layer is an adhesive layer or an injection molded plastic layer. In another aspect, the reaction layer is acrylic. In another aspect, the continuous circuitous channel has longitudinal/vertical and lateral/horizontal orientations. In another aspect, the device is configured to operate in a substantially vertical orientation aligned with gravity. In another aspect, the device is configured to operate via gravity-assisted capillary flow for sample transit through the microfluidic layer. In another aspect, the reaction layer has a thickness of 0.2 to 3.0 mm. In another aspect, the reaction chamber length ranges from about 10 mm to about 40 mm. In another aspect, the reaction chamber width ranges from about 2 mm to about 5 mm. In another aspect, the total channel length ranges from about 50 mm to about 600 mm. In another aspect, the channel width ranges from about 0.05 mm to about 2 mm. In another aspect, the channel thickness ranges from about 0.05 mm to about 0.5 mm. In another aspect, the residence time of a sample in the chamber ranges from about 5 minutes to about 2 hours. In another aspect, the residence time of a sample in the channel after it has emptied from the reaction chamber ranges from about 5 minutes to about 1 hours. In another aspect, the channel comprises from about 1 to 8 horizontal/lateral loops and 0 to 8 and vertical/longitudinal loops. In another aspect, the channel comprises one or a plurality of vertical/longitudinal oriented loops. In another aspect, the channel does not comprise any vertical/longitudinal oriented loops. In another aspect, the sample inlet delivers the sample directly into the lower reaction chamber. In another aspect, the sample inlet has a diameter from about 0.5 mm to about 1.5 mm. In another aspect, the sample inlet delivers the sample directly into the upper reaction chamber. In another aspect, the sample inlet has a diameter from about 1.0 mm to about 5.0 mm. In another aspect, the device further comprises an acrylic substrate layer on the same plane as the POEGMA substrate which creates a POEGMA-acrylic border. In another aspect, the device further comprises a bridge feature that allows the sample to cross the POEGMA-acrylic border on the bottom acrylic substrate layer at the edge of the POEGMA substrate without leaking. In another aspect, the device further comprises a tunnel feature built into the adhesive layers and the bottom acrylic substrate layer. In another aspect, the device further comprises a wash buffer delay channel. In another aspect, the device has functional improvements as compared to conventional devices, including: a longer shelf life; extended incubation times; room temperature storage and operation; low sample volume required for testing; capability of detecting multiple biomarkers simultaneously; and capability of being configured for multiple assay types with minor modifications to design.

Another embodiment described herein is the use of a device described herein for analyzing a biological sample by measuring a concentration level of an analyte in the biological sample.

Another embodiment described herein is a method for analyzing a biological sample by measuring a concentration level of an analyte, the method comprising: orienting a device described herein with gravity with the sample inlet at the top; loading a sample into the sample inlet; loading a wash buffer into the wash reservoir of the device; allowing the sample and wash buffer to enter and traverse completely through the device; imaging the device to measure a signal for the target analytes and controls; and determining the concentration of the analyte. In one aspect, the sample comprises whole blood, serum, plasma, urine, tears, sweat, saliva, lymph, cerebrospinal fluid, fecal extract, cellular or tissue extracts, or any other aqueous sample. In another aspect, the analyte is a biomolecule from an infectious agent, cancer, or is a biomarker for cardiovascular disease, or metabolic disorder. In another aspect, the analyte is a biomolecule or biomarker from a host response to an infectious agent, cancer, cardiovascular disease, or metabolic disorder. In another aspect, the analyte is a biomolecule or biomarker for SARS-CoV-2 or Ebola. In another aspect, the analyte is a cancer biomolecule or biomarker. In another aspect, the analyte is a biomolecule or biomarker associated with hepatocellular carcinoma.

Another embodiment described herein is a method for fabricating a microfluidic cassette assay device comprising a substrate layer having microfluidic geometry and a reagent layer disposed adjacent to the substrate layer, the method comprising: depositing a poly(oligoethylene glycol methyl ether methacrylate) (POEGMA) layer onto a glass substrate; depositing a trehalose layer upon the POEGMA layer; depositing a detection reagent onto the trehalose layers and a capture reagent onto the POEGMA layer at sites corresponding to a reaction chamber; adhering complementary layers of acrylic and adhesive sheets having microfluidic geometries onto the POEGMA substrate coated glass slide base, wherein the microfluidic geometries comprise a sample inlet, a wash reservoir, a reaction chamber comprising an upper chamber and a lower chamber separated by an offset mixing channel, a circuitous channel comprising a plurality of loops, and an outlet; and attaching a wash reservoir and absorbent waste pad. In one aspect, the acrylic and adhesive sheets are laser-cut to form microfluidic geometries prior to adhering onto the POEGMA substrate. In another aspect, the acrylic and adhesive sheets are injection molded to form microfluidic geometries prior to adhering onto the POEGMA substrate. In another aspect, the capture reagent and detection reagent are inkjet-printed and spatially separated to align with the corresponding microfluidic geometry of the substrate layer. In another aspect, the capture reagent is printed in the bottom region of the reaction chamber and the detection reagent is printed in the top region of the reaction chamber.

Another embodiment described herein is a microfluidic assay system comprising: a microfluidic assay device described herein; a stand; a wash buffer; and a sample applicator.

Another embodiment described herein is a kit comprising: a microfluidic assay device described herein; a stand; a wash buffer; and a sample applicator.

Another embodiment described herein is a multi-layered microfluidic assay device comprising a cassette comprising a plurality of layers comprising: a substrate layer comprising a non-fouling polymer layer coated on a glass substrate; and assay reagents disposed upon the substrate layer; a microfluidic layer comprising a channel layer and a reaction layer in fluid communication with each other and to inlets and outlets, the ultimate microfluidic layer adjacent to a cover layer; the channel layer comprising a continuous circuitous channel in fluid communication with the reaction layer; the reaction layer comprising cutouts for a reaction chamber, an inlet, and an outlet, each in fluid communication with the channel layer, the reaction layer being sandwiched between the channel layer on one side and the cover layer on an opposing side; and the reaction chamber comprising an upper reaction chamber, a lower reaction chamber, and an offset mixing channel fluidly connecting the upper and lower reaction chambers.

Another embodiment described herein is the use of a device described herein for analyzing a biological sample by measuring a concentration level of an analyte in the biological sample.

### DESCRIPTION OF THE DRAWINGS

FIGS. 1A-G show a DA-D4 point-of-care test (POCT) schematic and analytical validation. FIG. 1A shows a DA-D4 assay chip schematic where S1, RBD, and N capture antigens and fluorescently labeled S1 and N-NTD detection antigens (dAgs) are inkjet-printed onto a poly(oligoethylene glycol methyl ether methacrylate) (POEGMA) substrate. When a sample is added, dAgs are liberated from the surface due to the dissolution of the underlying trehalose pad. Antibodies targeting each viral antigen then bridge the capture antigens to the dAgs, resulting in a fluorescence signal that scales with antibody concentration. FIG. 1B shows an open format DA-D4 with 24 individual assays. FIG. 1C shows a microfluidic DA-D4. Sample is added to the sample inlet (SI), filling the reaction chamber (RC) which contains the assay reagents. Wash buffer is added to the wash buffer reservoir (WB) which chases the sample through the microfluidic cassette. The timing channel (TC) sets the residence time. All liquid is eventually soaked up by the wicking pad (WP) after the incubation process. The size is that of a standard microscope slide. FIG. 1D shows a D4Scope and cut-away view of the optical path. The microfluidic flow cell is inserted on the left, and pressing a button automates laser excitation, camera exposure, and data output. FIG. 1E shows analytical validation of the open format DAD4. Antibodies targeting each antigen were spiked into undiluted human serum and incubated for 30 min. Each data point represents the average of three independent runs, and the errors bars represent the standard error of mean (SEM). FIG. 1F shows analytical validation of microfluidic DA-D4. Each data point for an antigen represents the average of four independent microfluidic flow cells and error bars represent the SEM. FIG. 1G shows representative D4 spots for each dose.

FIGS. 2A-E show a DA-D4 print layout in open format and exemplary schematics of the microfluidic flow cell reaction chamber print format. FIG. 2A shows an open format print layout with a zoomed in view of individual arrays. For assays with 1P-10, an additional row of five capture antibody spots was printed and IP-10 detection antibody was added to the detection cocktail.

FIGS. 2B-C show the same exemplary schematic of a microfluidic chip reagent print format, with

FIG. 2B including labels. Three passes of trehalose pads were printed, followed by eight passes of the detection cocktail. All measurements listed in FIGS. 2A-B are in mm. FIG. 2D shows an exemplary illustration of the different layers for the detection region. The dashed line represents the addition of a sample 42. FIG. 2E shows an exemplary illustration of the different layers for the capture region following addition of a sample.

FIG. 3 shows an exemplary embodiment of an assembled cassette according to the present disclosure. These analysis components and features can be customized to provide optimal analysis for specific analytes. The embodiment shown in FIG. 3 can be used for antigen biomarker detection and double antigen serology assays in human serum, human plasma, or fetal bovine serum. The timing channels can be configured to achieve a particular residence time (e.g., 30 min). The exemplary device also includes an optional wash reservoir, which can be attached to or integrated with the cassette.

FIGS. 4A-B shows N-NTD purification. FIG. 4A shows that the N-terminal domain of the SARS-CoV-2 nucleocapsid protein (N-NTD) was expressed in E. coli and purified by IMAC, and aliquots from each step of the purification were visualized by SDS-PAGE. Lanes: ladder (lane 1), cell lysate (lane 2), clarified lysate (lane 3), IMAC flow through (lane 4), IMAC elution fractions (lanes 5-10). FIG. 4B shows comparison of detection reagents: full length N versus N-NTD in a double antigen assay. The response was examined to a high concentration sample (25 µg/mL) of anti-N antibody spiked into pooled human serum (black bars, n = 2) and a blank pooled human serum (grey bars, n = 6) sample, with standard deviations shown.

FIGS. 5A-D show exemplary schematics and layer assembly of a microfluidic flow cell. FIGS. 5A-B show the same detailed exploded schematic of all the layers and auxiliary attachments of the microfluidic flow cell, with FIG. 5A including labels. FIG. 5C shows a schematic showing vertical and horizontal orientations of the microfluidic flow cell and the dimension descriptions used herein. FIG. 5D shows a simplified schematic detailing the process flow for the laser-cutting and assembly of the individual layers of the microfluidic flow cell.

FIG. 6 shows an exploded view of the D4Scope. Files were rendered by SolidsWorks (Dassault Systems). All parts in grey are 3D printed in black poly-lactic acid plastic. M3 screws are used to fix all components in place: holding the D4 chip in focus relative to the camera/lens, enclosing the imaging axis (the path between the chip and the lens), and mounting the chip stage and Raspberry Pi (RPi) housing in place. M2 screws are used to mount the RPi to the housing.

FIGS. 7A-B show an analytical cross-reactivity study. FIG. 7A shows a graph of the measured D4 intensity with anti-S1/RBD antibodies spiked into undiluted, pooled human serum on open format DA-D4 chips at multiple concentrations. The response at each capture antigen was measured and plotted (n = 2) with error bars representing the standard deviations. FIG. 7B shows a graph of the measured D4 intensity with anti-N antibodies spiked into undiluted, pooled human serum on open format DA-D4 chips at multiple concentrations. The response at each capture antigen was measured and plotted (n = 2) with error bars representing the standard deviations.

FIG. 8 shows representative high positive (FIG. 8, left) and negative (FIG. 8, right) arrays from clinical patient samples. The diameter of each capture antigen is ~170 µm. Adjustments to image brightness and contrast were performed identically for both images.

FIGS. 9A-G show data from a clinical validation study. FIG. 9A shows the study design for COVID-19 ICU biorepository samples. Patients at Duke University Medical Center were enrolled into the study after admission to the ICU. Blood draws were taken at days 1, 3, 7, 14 and 21 after enrollment until discharge or death occurred. Aggregated data for 46 positive samples, 41 negative samples, and 18 acute/convalescent 229E (n = 2), HKU1 (n = 4), NL63 (n = 2), and OC43 (n = 10) samples tested for antibodies against S1 (FIG. 9B), RBD (FIG. 9C), and N (FIG. 9D). Dotted lines represent 2 SDs above the mean of the negative controls and the solid line represents the mean of each group. The box extends from the 25th to 75th percentiles and the line in the middle of the box is plotted at the median. The whiskers extend to the minimum and maximum values. Aggregated data was partitioned by days since symptom onset for S1 (FIG. 9E), RBD (FIG. 9F), and N (FIG. 9G). For five samples, the date since symptom onset was unknown, so days since first positive COVID-19 test were used (marked with an X).

FIG. 10 shows CAD renderings of microfluidic flow cells to illustrate the differences in flow cells used to test plasma/serum (FIG. 10, left) and whole blood (FIG. 10, right).

FIGS. 11A-C show an example of testing whole blood. FIG. 11A shows a modified microfluidic flow cell for testing whole blood. Zone 1: the reaction chamber was modified to prevent red blood cells from collecting in the chamber. Zone 2: The incubation timing channel was shortened to compensate for the slower flow rate of blood and to ensure blood did not clot or clog the channels. FIG. 11B shows a time lapse of blood and wash buffer in the reaction chamber.

FIG. 11C shows aggregated data for five positive samples and four negative controls tested for antibodies against S1, RBD, and N. Dotted lines represent 2 SDs above the mean of the negative controls. 100% sensitivity (5/5) and 100% specificity (4/4) were achieved for S1, RBD, and N.

FIGS. 12A-B show longitudinal antibody tracking. FIG. 12A shows antibody tracking for six patients across multiple time points for antibodies targeting S1, RBD, and N. For patient 3, date since symptom onset was unknown so days since first positive test was used. Each data point represents the average of two independent chips (with SD) run by separate users. FIG. 12B shows data from FIG. 12A for each repeat. The solid lines are drawn to have a slope of 1. There is a strong correlation between each repeat for S1, RBD, and N, with a Pearson’s r of 0.98, 0.97, and 0.97, respectively (p < 0.0001).

FIG. 13 shows correlation to microneutralization assays. Microneutralization assays were performed on each of the six different longitudinal patient samples from FIG. 12A (black diamonds). The log-transformed microneutralization titer is plotted on the left axis superimposed against the antibody data from FIG. 12A (plotted on the right axis).

FIG. 14 shows DA-D4 readout correlation with microneutralization assays. Correlations are shown between anti-S1 (FIG. 14, left), anti-RBD (FIG. 14, center), and anti-N (FIG. 14, right) with microneutralization assay for a subset of 34 patient samples from FIGS. 9 with Pearson r reported.

FIGS. 15A-D show combined prognostic biomarker and serology detection. FIG. 15A shows a dose-response curve for recombinant IP-10 spiked into FBS. Each data point represents the average (n = 3) and error bars represent the SEM. The limit-of-detection (LOD) for IP-10 is 0.12 ng/mL. FIG. 15B shows a dose-response curve for anti-SARS-CoV-2 antibodies spiked into FBS. The highest concentration is 10 µg/mL of anti-S1/RBD and 10 µg/mL of anti-N antibodies. Each data point represents the average (n = 3) with SEM. FIG. 15C shows the correlation between DA-D4 readout for IP-10 with an ELISA assay performed separately. Samples with a letter designate samples from one individual at different time points, where “b” occurs later in disease than “a.” All samples were tested in duplicate on the DA-D4 (with SD shown) except 2b due to insufficient volume. The solid line shows linear regression. FIG. 15D shows antibody reactivity against S1, RBD, and N for sample tested in FIG. 15C (with SD shown). NC = negative control pooled healthy plasma.

FIGS. 16A-C show data for multiplexed D4 for COVID-19 prognosis. Analytical dose response curves for three potential prognostic biomarkers are shown: NT-proBNP (FIG. 16A), D-dimer (FIG. 16B), and IL-6 (FIG. 16C). Capture and detection antibodies for each biomarker were inkjet-printed onto D4 chips and each antigen was spiked into FBS at physiological relevant concentrations. Dose response curves were run in triplicate with SEM shown.

FIG. 17 shows microfluidic D4 cassette dAb titration optimization for ovalbumin. The error bars shown represent the standard error of the mean.

FIGS. 18A-B show microfluidic dose response curves in FBS imaged on the D4Scope (FIG. 18A) and Genepix scanner (FIG. 18B). The error bars shown represent the standard error of the mean.

FIGS. 19A-B show microfluidic D4 cassette spike and recovery results collected on the D4Scope (FIG. 19A) and Genepix scanner (FIG. 19B).

FIGS. 20A-D show microfluidic D4 cassette responses to varied temperature levels imaged on the D4Scope (FIG. 20A) and Genepix scanner (FIG. 20B), and to varied humidity levels imaged on D4Scope (FIG. 20C) and Genepix scanner (FIG. 20D). The error bars shown represent the standard error of the mean.

FIGS. 21A-B show microfluidic D4 cassette response to pooled human urine imaged on the D4Scope (FIG. 21A) and Genepix scanner (FIG. 21B). The error bars shown represent the standard error of the mean.

FIGS. 22A-B show microfluidic D4 cassette response to varied hematocrit percentage in single donor human whole blood imaged on the D4Scope (FIG. 22A) and Genepix scanner (FIG. 22B). The error bars shown represent the standard error of the mean.

FIG. 23 shows a multiplexed panel for detection of HCC biomarkers (n = 4). The error bars shown represent the standard error of the mean.

FIGS. 24A-I show 2D schematics illustrating the orientations of the reaction chamber, timing channels, inlets, and outlets for the Original, Original Blood, and alternative A1-A5 cassette designs. FIG. 24A shows a side-by-side comparison view of the Original, Original Blood, and alternative A1-A5 cassette designs. FIG. 24B shows an enlarged side-by-side comparison view of the Original and Original Blood cassette designs. FIG. 24C shows an enlarged view of the alternative A1 cassette design. FIG. 24D shows an ovalbumin dose response curve analyzed on the Genepix scanner (FIG. 24D, left) and on the D4Scope (FIG. 24D, right). FIG. 24E shows an enlarged view of the alternative A2 cassette design. FIG. 24F shows an enlarged view of the alternative A3 cassette design. FIG. 24G shows an enlarged view of the alternative A4 multiplexed cassette design. FIG. 24H shows an ovalbumin dose response curve (FIG. 24H, left) conducted in spiked FBS and analyzed on the Genepix scanner that compares performance of the A4 device (blue, n = 4) with the original version of the device (red, n = 4). An sGP dose response curve (FIG. 24H, right) was conducted in spiked human serum and analyzed on the Genepix Scanner (n = 4). FIG. 24I shows an enlarged view of the alternative A5 cassette design.

FIG. 25 shows a graph demonstrating the extended residence time that results from the vertical loops of the timing channels. The red line indicates the residence time in the vertical loops of the timing channel, while the blue line indicates the residence time in the downstream portions of the channel. The velocity of the fluid front (displacement/time) increases once the fluid front moves past the vertical loops into the downstream portions.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising,” The present disclosure also contemplates other embodiments “comprising,” “consisting of,” and “consisting essentially of,” the embodiments or elements presented herein, whether explicitly set forth or not

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ± 10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 ... 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “effective amount” or “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject’s age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The term “expression vector” indicates a plasmid, a virus or another medium, known in the art into which a nucleic acid sequence for encoding a desired protein can be inserted or introduced.

The term “host cell” is a cell that is susceptible to transformation, transfection, transduction, conjugation, and the like with a nucleic acid construct or expression vector.

Host cells can be derived from plants, bacteria, yeast, fungi, insects, animals, etc. In some embodiments, the host cell includes Escherichia coli.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. Alternatively, cutoff values may be determined by a quartile analysis of biological samples of a patient group. For example, a cutoff value may be determined by selecting a value that corresponds to any value in the 25^{th}-75^{th} percentile range, preferably a value that corresponds to the 25^{th} percentile, the 50^{th} percentile or the 75^{th} percentile, and more preferably the 75^{th} percentile. Such statistical analyses may be performed using any method known in the art and can be implemented through any number of commercially available software packages. The healthy or normal levels or ranges for a target or for a protein activity may be defined in accordance with standard practice.

“Polynucleotide” as used herein can be single stranded or double stranded or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide,” “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tall domains, “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length, in some embodiments, a motif includes 3, 4, 5, 6, or 7 sequential amino acids. A domain may be comprised of a series of motifs, which may be similar or different.

“Recombinant” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all.

The term “host cell” is a cell that is susceptible to transformation, transfection, transduction, conjugation, and the like with a nucleic acid construct or expression vector. Host cells can be derived from plants, bacteria, yeast, fungi, insects, animals, etc. In some embodiments, the host cell includes Escherichia coli.

“Sample” or “test sample” as used herein can mean any sample in which the presence and/or level of a target is to be detected or determined or any sample comprising an agent, cell, or partially ordered polypeptide as described herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pretreated, such as by filtration, distillation, extraction, concentration, centrifugation, inactivation of interfering components, addition of reagents, and the like, to modify the character of the sample in some manner as discussed herein or otherwise as is known in the art.

As used herein, the term “biomarker” refers to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration of a biomarker, can be associated with and/or be indicative of a particular state or process. Biomarkers can include, but are not limited to, cells or cellular components (e.g., a viral cell, a bacterial cell, a fungal cell, a cancer cell, etc.), small molecules, lipids, carbohydrates, nucleic acids, peptides, proteins, enzymes, antigens, and antibodies. A biomarker can be derived from an infectious agent, such as a bacterium, fungus or virus, or can be an endogenous molecule that is found in greater or lesser abundance in a subject suffering from a disease or disorder as compared to a healthy individual (e.g., an increase or decrease in expression of a gene or gene product).

“Polymer” as used herein is intended to encompass a homopolymer, heteropolymer, block polymer, co-polymer, ter-polymer, etc., and blends, combinations, or mixtures thereof. Examples of polymers include, but are not limited to, functionalized polymers, such as a polymer comprising 5-vinyltetrazole monomer units and having a molecular weight distribution less than 2.0. The polymer may be or contain one or more of a star block copolymer, a linear polymer, a branched polymer, a hyperbranched polymer, a dendritic polymer, a comb polymer, a graft polymer, a brush polymer, a bottle-brush copolymer and a crosslinked structure, such as a block copolymer comprising a block of 5-vinyltetrazole monomer units. Polymers include, without limitation, polyesters, poly(meth)acrylamides, poly(meth)acrylates, polyethers, polystyrenes, polynorbornenes and monomers that have unsaturated bonds. For example, amphiphilic comb polymers are described in U.S. Pat. Application Publication No. US 2007/0087114 and in U.S. Pat. No. 6,207,749 to Mayes et al., the disclosure of each of which is herein incorporated by reference in its entirety. The amphiphilic comb-type polymers may be present in the form of copolymers, containing a backbone formed of a hydrophobic, water-insoluble polymer and side chains formed of short, hydrophilic non-cell binding polymers. Examples of other polymers include, but are not limited to, polyalkylenes such as polyethylene and polypropylene-, polychloroprene; polyvinyl ethers; such as polyvinyl acetate); polyvinyl halides such as polyvinyl chloride); polysiloxanes; polystyrenes; polyurethanes; polyacrylates; such as poly(methyl (meth)acrylate), poly(ethyl (meth)acrylate), poly(n-butyl(meth)acrylate), poly(isobutyl (meth)acrylate), poly(tert-butyl (meth)acrylate), poly(hexyl(meth)acrylate), poly(isodecyl (meth)acrylate), poly(lauryl (meth)acrylate), poly(phenyl (meth)acrylate), poly(methyl acrylate), poly(isopropyl acrylate), poly(isobutyl acrylate), and poly(octadecyl acrylate); polyacrylamides such as poly(acrylamide), poly(methacrylamide), poly(ethyl acrylamide), poly(ethyl methacrylamide), poly(N-isopropyl acrylamide), poly(n, iso, and tert-butyl acrylamide), and copolymers and mixtures thereof. These polymers may include useful derivatives, including polymers having substitutions, additions of chemical groups, for example, alkyl groups, alkylene groups, hydroxylations, oxidations, and other modifications routinely made by those skilled in the art. The polymers may include zwitterionic polymers such as, for example, polyphosphorycholine, polycarboxybetaine, and polysulfobetaine. The polymers may have side chains of betaine, carboxybetaine, sulfobetaine, oligoethylene glycol (OEG), sarcosine, or polyethyleneglycol (PEG). For example, poly(oligoethyleneglycol methacrylate) (poly(OEGMA)) may be used. Poly(OEGMA) may be hydrophilic, water-soluble, non-fouling, non-toxic and non-immunogenic due to the OEG side chains.

As used herein, the term “region” refers to a defined area on the surface of a material. A region can be identified and bounded by a distinct interface between two materials having different compositions.

“Specific binding pair” as used herein refers to two molecules that exhibit specific binding to one another, or increased binding to one another relative to other molecules. A specific binding pair can exhibit functional binding activity such as, for example, a receptor and a ligand (such as a drug, protein, or carbohydrate), an antibody and an antigen, etc.; or structural binding activity such as, for example, protein/peptide and protein/peptide; protein/peptide and nucleic acid; and nucleotide and nucleotide etc. Typically, one member of the binding pair can serve as a capture agent in the devices described herein, and the capture agent can bind to the second member of the binding pair, which can be present as an analyte in a sample such as a biological fluid. “Analyte” as used herein can be any second member of a specific binding pair, as described above. Typically, the analyte is a constituent of, or found in, a sample such as a biological fluid. The analyte can be a biomarker as described above.

“Polynucleotide” as used herein can be single stranded or double stranded or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide,” “protein,” and “peptide” are used interchangeably herein. “Primary structure” refers to the amino acid sequence of a particular peptide. “Secondary structure” refers to locally ordered, three-dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units.

“Reporter,” “reporter group,” “label,” and “detectable label” are used interchangeably herein. The reporter is capable of generating a detectable signal. The label can produce a signal that is detectable by visual or instrumental means. A variety of reporter groups can be used, differing in the physical nature of signal transduction (e.g., fluorescence, electrochemical, nuclear magnetic resonance (NMR), and electron paramagnetic resonance (EPR)) and in the chemical nature of the reporter group. Various reporters include signal-producing substances, such as chromagens, fluorescent compounds, chemiluminescent compounds, radioactive compounds, and the like. In some embodiments, the reporter comprises a radiolabel. Reporters may include moieties that produce light, e.g., acridinium compounds, and moieties that produce fluorescence, e.g., fluorescein. In some embodiments, the signal from the reporter is a fluorescent signal. The reporter may comprise a fluorophore. Examples of fluorophores include, but are not limited to, acrylodan (6-acryloy 1-2-dimethylaminonaphthalene), badan (6-bromo-acetyl-2-dimethylamino-naphthalene), rhodamine, naphthalene, dansyl aziridine, 4-[N-[(2-iodoacetoxy)ethyl]-N-methylamino]-7-nitrobenz-2-oxa-1,3-diazole ester (IAN BDE), 4-[N-[(2-iodoacetoxy)ethyl]-N-methylamino-7-nitrobenz-2-oxa-1,3-diazole (IAN BDA), fluorescein, dipyrrometheneboron difluoride (BODIPY), 4-nitrobenzo[c][1,2,5]oxadiazole (NBD), Alexa fluorescent dyes, and derivatives thereof. Fluorescein derivatives may include, for example, 5-fluorescein, 6-carboxyfluorescein, 3′-6-carboxyfluorescein, 5(6)-carboxyfluorescein, 6-hexachlorofluorescein, 6-tetrachlorofluorescein, fluorescein, and isothiocyanate.

As used herein, the term “detection moiety” is any moiety or compound that is detectable by methods including, but not limited to, spectroscopic, photochemical, biochemical, immunochemical, chemical, electrochemical, radioactivity, and other physical means. A detection moiety can be detectable indirectly; for example, the detection moiety can be a moiety or compound that is a member of a specific binding pair, wherein the second member of the binding pair can include a detection moiety that can be detected directly. A non-limiting and known example of such a detection moiety is biotin, which can bind to avidin or streptavidin comprising a detection moiety such as a fluorophore. Exemplary detection moieties can include, but are not limited to, fluorophores, chromophores, radiolabels, polynucleotides, small molecules, enzymes, nanoparticles, and upconverters.

“Sample” or “test sample” as used herein can mean any sample in which the presence and/or level of a target is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pretreated, such as by filtration, distillation, extraction, concentration, centrifugation, inactivation of interfering components, addition of reagents, and the like, to modify the character of the sample in some manner as discussed herein or otherwise as is known in the art.

The term “sensitivity” as used herein refers to the number of true positives divided by the number of true positives plus the number of false negatives, where sensitivity (“sens”) may be within the range of 0 < sens < 1. Ideally, method embodiments herein have the number of false negatives equaling zero or close to equaling zero, so that no subject is wrongly identified as not having a disease when they indeed have the disease. Conversely, an assessment often is made of the ability of a prediction algorithm to classify negatives correctly, a complementary measurement to sensitivity.

The term “specificity” as used herein refers to the number of true negatives divided by the number of true negatives plus the number of false positives, where specificity (“spec”) may be within the range of 0 < spec < 1. Ideally, the methods described herein have the number of false positives equaling zero or close to equaling zero, so that no subject is wrongly identified as having a disease when they do not in fact have disease. Hence, a method that has both sensitivity and specificity equaling one, or 100%, is preferred.

By “specifically binds,” it is generally meant that a polypeptide binds to a target when it binds to that target more readily than it would bind to a random, unrelated target.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, rats, mice, rabbits, pigs, cows, sheep, goats, horses, dogs, cats, fish, birds, and the like. Typically, the subject is a mammal. A subject also refers to primates (e.g., humans, male or female; infant, adolescent, or adult) or non-human primates. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

“Transition” or “phase transition” refers to the aggregation of the thermally responsive polypeptides. Phase transition occurs sharply and reversibly at a specific temperature called the lower critical solution temperature (LCST) or the inverse transition temperature T^, Below the transition temperature, the thermally responsive polypeptide (or a polypeptide comprising a thermally responsive polypeptide) is highly soluble. Upon heating past the transition temperature, the thermally responsive polypeptides hydrophobically collapse and aggregate, forming a separate, gel-like phase. “Inverse transition cycling” refers to a protein purification method for thermally responsive polypeptides (or a polypeptide comprising a thermally responsive polypeptide). The protein purification method may involve the use of thermally responsive polypeptide’s reversible phase transition behavior to cycle the solution through soluble and insoluble phases, thereby removing contaminants.

“Treatment” or “treating,” when referring to protection of a subject from a disease, means preventing, suppressing, repressing, ameliorating, or eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

The terms “residence time” or “incubation time” are used interchangeably herein. These times refer to the any amount of time a sample takes to traverse within the microfluidic device described herein, for example the time of transit from the inlet to the outlet. These terms can be used to describe discrete intervals of time such as the amount of time a sample spends in the reaction area or chamber.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or greater number of amino acids.

“Valency” as used herein refers to the potential binding units or binding sites. The term “multivalent” refers to multiple potential binding units. The terms “multimeric” and “multivalent” are used interchangeably herein.

“Variant” used herein with respect to a polynucleotide means (i) a portion or fragment of a referenced nucleotide sequence; (ii) the complement of a referenced nucleotide sequence or portion thereof, (iii) a polynucleotide that is substantially identical to a referenced polynucleotide or the complement thereof, or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids. See Kyte et al., J. Mol. Biol, 1982, 757, 105-132. The hydropathic index of an amino acid is based on a consideration of its hydrophobicity and charge. It is known in the art that amino acids of similar hydropathic indexes can be substituted and retain protein function. In one aspect, amino acids having hydropathic indices of ± 2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S.

Pat. No. 4,554,101, which is incorporated herein by reference. Substitution of amino acids having similar hydrophilicity values can result in polypeptides retaining biological activity, for example immunogenicity, as is understood in the art. Substitutions can be performed with amino acids having hydrophilicity values within ± 2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 88%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

### Polymer Layer

The polymer layers of the devices described herein exhibit non-fouling properties. Non-fouling, as used herein with respect to the polymer layer, relates to the inhibition (e.g., reduction or prevention) of growth of an organism as well as to non-specific or adventitious binding interactions between the polymer and an organism or biomolecule (e.g., cell, protein, nucleotide, etc.). The non-fouling property of the polymer can be introduced by any suitable method such as, for example, incorporation of a non-fouling (or alternatively, antifouling) agent or by the structure/architecture of the polymer itself. Non-fouling agents are known in the art and can be selected by one of skill depending on the particular use of device, or on the availability of the non-fouling agent. Non-limiting examples can include organic and inorganic compounds having biocidal activity, as well as compounds that can be incorporated with or bound to the polymer layer that reduce or inhibit non-specific binding interaction of a biomolecule (e.g., cell, protein, nucleotide, carbohydrate/lipid) with the polymer upon contact.

Some embodiments provide a polymer layer having a structure or architecture that provides a non-fouling property. In some of the embodiments described herein, the polymer can suitably include brush polymers, which are, in general, formed by the polymerization of monomelic core groups having one or more groups that function to inhibit binding of a biomolecule (e.g., cell, protein, nucleotide, carbohydrate/lipid) coupled thereto. Suitably, the monomelic core group can be coupled to a protein-resistant head group.

Polymer layers can suitably be formed using radical polymerization techniques, such as catalytic chain transfer polymerization, iniferter mediated polymerization (e.g., photoiniferter mediated polymerization), free radical polymerization, stable free radical mediated polymerization (SFRP), atom transfer radical polymerization (ATRP), and reversible addition- fragmentation chain transfer (RAFT) polymerization.

For example, free radical polymerization of monomers to form brush polymers can be carried out in accordance with known techniques, such as described in U.S. Pat. Nos. 6,423,465; 6,413,587; and 6,649,138; U.S. Pat. Application Publication No. US 2003/0108879 A1, and variations thereof which will be apparent to those skilled in the art. Atom transfer radical polymerization of monomers to form brush polymers can also be carried out in accordance with known techniques, such as described in U.S. Pat. Nos. 6,541,580 and 6,512,060; U.S. Pat. Application Publication No. US 2003/0185741 A1, and variations thereof which will be apparent to those skilled in the art.

Any suitable core vinyl monomer polymerizable by the processes discussed above can be used, including but not limited to styrenes, acrylonitriles, acetates, acrylates, methacrylates, acrylamides, methacrylamides, vinyl alcohols, vinyl acids, and combinations thereof.

In some of the embodiments described herein, the polymer layer can be formed by surface-initiated ATRP (Sl-ATRP) of oligo(ethylene glycol)methyl methacrylate (OEGMA) to form a poly(OEGMA) (POEGMA) film. In an embodiment, the polymer layer is a functionalized POEGMA film prepared by copolymerization of a methacrylate and methoxy terminated OEGMA. Suitably, the POEGMA polymer can be formed in a single step.

In general, the brush molecules formed by the processes described herein (or other processes either known in the art or which will be apparent to those skilled in the art), can be from 2 or 5 up to 100 or 200 nanometers in length, or more, and can be deposited on the surface portion at a density of from 10, 20, or 40 to up to 100, 200 or 500 milligrams per meter, or more.

Protein resistant groups can be hydrophilic head groups or kosmotropes. Examples can include but are not limited to oligosaccharides, tri(propyl sulfoxide), hydroxyl, glycerol, phosphorylcholine, tri(sarcosine) (Sarc), N-acetylpiperazine, betaine, carboxybetaine, sulfobetaine, permethylated sorbitol, hexamethylphosphoramide, an intramolecular zwitterion (for example, —CH₂N⁺(CH₃)₂CH₂CH₂CH₂SO₃) (ZW), and mannitol.

Additional examples of kosmotrope protein resistant head groups can comprise:
—(OCH₂CH₂)₆OH;
-O(Mannitol);
—C(O)N(CH₃)CH₂(CH(OCH₃))₄CH₂OCH₃;
—N(CH₃)₃⁺Cl⁻/—SO₃⁻Na⁺(1—1);
—N(CH₃)₂ +CH₂CH₂SO₃;
-C(O)Pip(NAc) (Pip=piperazinyl);
—N(CH₃)₂ +CH₂CO₂;
-O([Gle-α(1,4)-Glc-β(1)⁻]);
—C(O)(N(CH₃)CH₂C(O))₃N(CH₃)₂;
—N(CH₃)₂⁺CH₂CH₂CH₂SO₃ ⁻;
—C(O)N(CH₃)CH₂CH₂N(CH₃)P(O)(N(CH₃)₂)₂⁻; or
—S(O)CH₂CH₂CH₂)₃S(O)CH₃.

In some of the embodiments described herein, a suitable protein resistant head group can comprise poly(ethylene glycol) (PEG), for example PEG of from 3 to 20 monomelic units.

Prior to deposition of further components onto the polymer layer, the substrate with the optional linking layer and polymer layer can be dry or at least macroscopically dry (that is, dry to the touch or dry to visual inspection, but retaining bound water or water of hydration in the polymer layer). For example, to enhance immobilization of a capture agent, the polymer layer can suitably retain bound water or water of hydration, but not bulk surface water. If the substrate with the linking layer and polymer layer has been stored in desiccated form, bound water or water of hydration can be reintroduced by quickly exposing the polymer layer to water (e.g., by dipping in to water) and subsequently blow-drying the surface (e.g., with a nitrogen or argon jet). Alternatively, bound water or water of hydration can be reintroduced by exposing the polymer layer to ambient air for a time sufficient for atmospheric water to bind to the polymer layer.

In one embodiment, the polymer layer is disposed on a substrate. In one aspect, the substrate layer is glass. In one aspect, the polymer layer is a poly(oligoethylene glycol methyl ether methacrylate) (POEGMA).

### Microfluidic Layer/Cover Layer

In one embodiment, the microfluidic layers or cover layers comprise adhesive or hydrophilic polymer sheets comprising one or more of polyacrylic acid, polymethylmethacrylate, polycarbonate, polyester, nylon, polyvinyl chloride, polyethylene, polypropylene, polyethylene terephthalate glycol, polybutylene adipate terephthalate, ethylene tetrafluoroethylene, fluorinated ethylene propylene, perfluoro alkoxy alkane, polylactic acid, polycaprolactone, polyoxymethylene, cellulose, co-polymers thereof, or combinations thereof.

In one embodiment, the microfluidic layers are fabricated to create microfluidic channels, reservoirs, reaction areas, inlets and outlets by one or more of laser cutting, injection molding, die cutting, milling, press cutting, layer-by-layer fabrication, 3D printing, lithography, or combinations thereof.

In one embodiment, the microfluidic layer comprises at least two layers comprising an adhesive layer comprising microfluidic channels, reservoirs, reaction areas, inlets, and outlets and a reaction layer comprising a hydrophilic polymer having reaction areas, inlets, and outlets. When viewed in a horizontal orientation, the microfluidic layers are sandwiched between a base substrate layer comprising non-fouling polymer disposed on a glass substrate and a cover layer comprising a hydrophilic polymer sheet comprising inlets, and outlets.

### Reagents

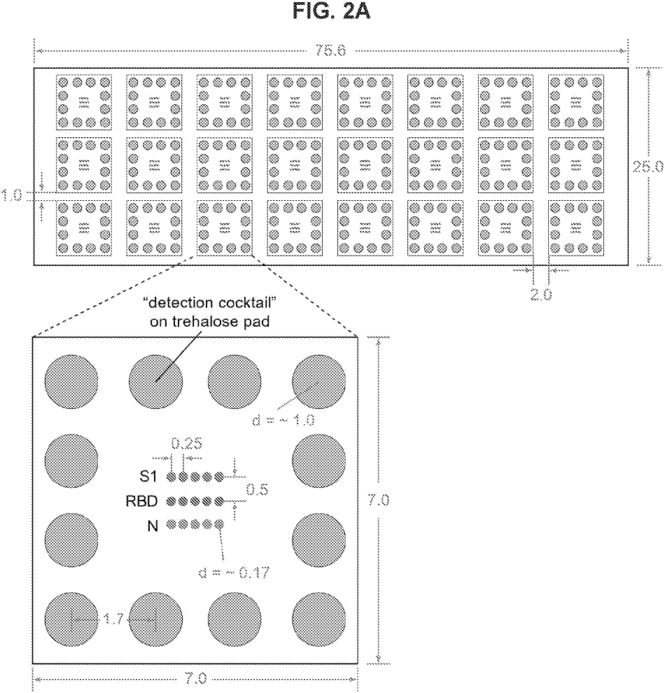

As described herein, “reagents” refer to “capture arrays,” “capture reagents,” “capture antigens,” “detection reagents,” “detection antigens,” and “controls” that are deposited on a non-fouling polymer layer spatially separated to align with the reaction chambers of the microfluidic layers. In some embodiments, the capture arrays comprise capture antigens and the detection reagents comprise fluorescently labeled detection antigens (dAgs) disposed on trehalose “pads.” See FIGS. 2A-B. Both the capture arrays and detection reagents are printed onto the non-fouling polymer (e.g., (POEGMA) disposed on a substrate. When a sample is added, the detection antigens (dAg) are liberated from the surface due to the dissolution of the underlying trehalose pad. Antibodies targeting each antigen then bridge the capture antigens to the dAgs, resulting in a fluorescence signal that scales with antibody concentration.

FIG. 2C shows an exemplary embodiment of a reaction chamber (2) having reagent layers printed on a POEGMA coated glass slide. A sample inlet (4) provides sample to a bottom region (14) of the reaction chamber (2). Wash buffer enters a top region (8) of the reaction chamber (2) through a wash reservoir inlet (6) and dissolves a plurality of attached trehalose pads (10), thereby liberating the disposed detection antigens. The liberated detection antigens move through an offset (12) and enter the bottom region (14) of the reaction chamber (2). A capture array region (16) comprising capture antigens is printed directly on the POEGMA in the bottom region (14) along with control spot regions (18). A reaction chamber outlet (20) allows the sample mixture to exit the bottom region (14) of the reaction chamber (2) and enter the timing channels of the microfluidic device.

FIGS. 2D-E show an exemplary embodiment of the stacking for the detection and capture reagent layers on a POEGMA coated glass slide. In FIG. 2D, a detection reagent layer (30) is comprised of a glass slide base (32) coated with a POEGMA layer (34) deposited with a plurality of trehalose pads (36). Detection antigens (38) are printed on the plurality of trehalose pads (36) to produce a detection reagent layer (30). The dashed line indicates the addition of a target sample (40). In FIG. 2E, a capture reagent layer (50) is comprised of a glass slide base (52) coated with a POEGMA layer (54) printed with a capture antigen array (56). The capture antigen array (56) binds to a detection-sample complex (62) comprised of detection antigens (58) that are bound to a target sample (60).

### Multi-Layer Microfluidic Device

### Nomenclature

The layer-by-layer description and assembly of the multi-layer microfluidic devices are described herein with reference to the Horizontal and Vertical Orientations as shown in FIG. 5C. The same descriptors are used for both the Vertical and Horizonal orientations for consistency. The descriptors used are: Vertical/Longitudinal (Length) - spanning the longest dimension of the rectangular plane; Horizontal/Lateral (Width) - spanning the shorter dimension of the rectangular plane; and Thickness/Transverse (Height) - spanning the vertical dimension of the rectangular plane when viewed in the Horizontal orientation or the horizontal dimension when viewed in the Vertical orientation. When viewed in the Horizontal orientation, the “bottom layer” is the substrate layer and the “top layer” is the cover layer. When describing the “length” of the circuitous channel, this is the end-to-end straightened length of the channel. The thickness or height describes the dimension of the channel in which liquid flows.

### Description

One embodiment described herein is a multi-layer, microfluidic device, comprising: a plurality of layers. In one aspect, when viewed in a Horizontal orientation (see FIG. 5C), the bottom substrate layer comprises a glass layer coated with a non-fouling polymer. Reagents deposited on the non-fouling polymer layer are spatially separated to align with the reaction areas of the microfluidic layers. The microfluidic layer comprises an adhesive or hydrophilic polymer layer and a reaction layer. In one aspect, sandwiched between the substrate layers with a non-fouling polymer layer hydrophilic polymer layer and the reaction layer, is an adhesive layer or hydrophilic polymer layer containing cutouts for channels, reaction areas, inlets and outlies that provide fluid communication with the reaction layer. The reaction layer contains a plurality of spaces for the reaction areas. In another aspect, the microfluidic layer is a single layer comprising an injection molded polymer comprising both the channels and cutouts for the reaction area. Multiple multi-layer, microfluidic devices can be assembled on a single substrate to provide a manifold of separate or interconnected microfluidic devices.

One embodiment described herein is a multi-layered microfluidic device cassette 90, comprising: a plurality of uniquely stacked layers (100, 110, 120, 190, 240, 280) with two or more additional attachments including a wash reservoir and absorbent pad (310, 320). A glass slide (100) is coated with a POEGMA polymer layer (110) to form a base substrate that serves as the base for the microfluidic channels. A first adhesive layer (120) covers the base substrate and comprises a sample inlet (130), wash chamber (140), reaction chamber (comprising an upper and lower reaction chamber) (150), vertical and horizontal timing channels (continuous circuitous channel) (160, 170), and outlet opening (180) in contact with an absorbent pad. The outline of the reaction chamber (150) and wash chamber (140) are left exposed to allow the sample and wash buffer to contact the POEGMA substrate. A first acrylic layer (190) covers the first adhesive layer (120) and provides a front wall of the microfluidic channels, enclosing them on all four sides. The first acrylic layer (190) comprises cutouts for the sample inlet (200), wash chamber (210), reaction chamber (comprising an upper and lower reaction chamber) (220), outlet opening (230) and absorbent pad. A second adhesive layer (240) covers the first acrylic layer (190) and primarily serves as a seal between acrylic layers. The second adhesive layer (240) comprises cutouts for a sample inlet (250), wash chamber (260), and reaction chamber (270). The outline of the reaction chamber (270) ensures there is an optically transparent path for the excitation laser to travel through during imaging, preventing scattering of laser light on the less transparent adhesive that could impact performance. The outline of the wash chamber (260) provides access to the wash inlet needed for cassette operation. A second acrylic layer (280) covers the second adhesive layer (240) and seals the reaction chamber as the top layer. The second acrylic layer (280) has cutouts for the sample inlet (290) and wash chamber (300). A 3D-printed wash reservoir (310) capable of holding up to 250 µL of wash buffer is attached to the surface of the cover layer and is in fluid communication with the wash chamber (300). A laser-cut absorbent pad (320) is in fluid communication with the outlet and absorbs waste from the cassette; the absorbent pad is held in place with an acrylic wash cover to reduce exposure of the waste to the end user. See FIGS. 5A-C.

Another embodiment described herein is a multi-layered microfluidic assay device (90) comprising a cassette comprising a plurality of layers (100, 110, 120, 190, 240, 280) comprising: a substrate layer comprising a non-fouling polymer layer (110) coated on a glass substrate (100); and assay reagents disposed (38, 50) upon the substrate layer; a microfluidic layer (120, 190) comprising a channel (160) and a reaction area (150, 220) in fluid communication with each other and to inlets (130, 150, 200, 210) and outlets (180, 230), the ultimate microfluidic layer (e.g., the reaction layer) (190) adjacent to a cover layer (280) and optionally affixed to the cover layer (280) via an adhesive layer (240) having cutouts for the reaction chamber (270), and inlets (250, 260); the microfluidic layer comprising a channel layer (120) comprising a continuous circuitous channel (160) and a reaction area (150) in fluid communication with a reaction layer (190), the reaction layer (190) comprising cutouts for a reaction chamber (220), inlets (200, 220), and an outlet (230), each in fluid communication with the channel layer (120), the reaction layer being sandwiched between the channel layer (120) on one side and the cover layer (280) on the other side; the reaction chamber (150, 220, 270) comprising an upper reaction chamber (8), a lower reaction chamber (14), and an offset mixing channel (12) fluidly connecting the upper (8) and lower (14) reaction chambers. In another embodiment, the microfluidic layer is a single layer comprising an injection molded polymer comprising both the channels and cutouts for the reaction area.

Another embodiment described herein is a multi-layered microfluidic assay device (90) comprising a cassette comprising a plurality of layers (100, 110, 120, 190, 240, 280) comprising: a substrate layer comprising a non-fouling polymer layer (110) coated on a glass substrate (100); and assay reagents disposed (38, 50) upon the substrate layer; a microfluidic layer (120, 190) comprising a channel layer (120) and a reaction layer (190) in fluid communication with each other and to inlets (130, 150, 200, 210) and outlets (180, 230), the reaction layer (190) affixed to a cover layer (280) via an adhesive layer (240) having cutouts for the reaction chamber (270), and inlets (250, 260); the channel layer (120) comprising a continuous circuitous channel (160) and a reaction area (150) in fluid communication with a reaction layer (190); the reaction layer (190) comprising cutouts for a reaction chamber (220), inlets (200, 220), and an outlet (230), each in fluid communication with the channel layer (120), the reaction layer (190) being sandwiched between the channel layer (120) on one side and the cover layer (280) on an opposing side via an adhesive layer (240); and the reaction chamber (150, 220, 270) comprising an upper reaction chamber (8), a lower reaction chamber (14), and an offset mixing channel (12) fluidly connecting the upper (8) and lower (14) reaction chambers. In another embodiment, the microfluidic layer is a single layer comprising the channel layer and reaction layer as an injection molded polymer comprising both the channels and cutouts for the reaction area. In one aspect, the offset mixing channel comprises a P-trap bend to prevent clogging. In another aspect, the assay reagents comprise one or more detection reagents and one or more capture reagents that are spatially separated and disposed on the non-fouling polymer layer to align with the lower and upper reaction chambers. In another aspect, the detection reagents are placed on layers of trehalose, disposed on the substrate spatially at a situs corresponding to the upper reaction chamber; and of the capture reagents are disposed on the substrate spatially at a situs corresponding to the lower reaction chamber. In another aspect, the cover layer is attached to the reaction layer via an adhesive layer. In another aspect, the channel layer, the reaction layer, and the cover layer are interconnectedly vented to ambient atmospheric pressure. In another aspect, the cover layer comprises one or more inlets and outlets in fluid communication with the microfluidic layers and with a reservoir at the inlets and an absorbent waste pad at the outlets. In another aspect, the non-fouling polymer layer is a poly(oligoethylene glycol methyl ether methacrylate) (POEGMA). In another aspect, the channel layer is an adhesive layer or an injection molded polymethylmethacrylate layer. In another aspect, the reaction layer is acrylic. In another aspect, the continuous circuitous channel has longitudinal/vertical and lateral/horizontal orientations. In another aspect, the device is configured to operate in a substantially vertical orientation aligned with gravity. In another aspect, the device is configured to operate via gravity-assisted capillary flow for sample transit through the microfluidic layer. In another aspect, the reaction layer has a thickness of 0.2 to 3.0 mm. In another aspect, the reaction chamber length ranges from about 10 mm to about 40 mm. In another aspect, the reaction chamber width ranges from about 2 mm to about 5 mm. In another aspect, the total channel length ranges from about 50 mm to about 600 mm. In another aspect, the channel width ranges from about 0.05 mm to about 2 mm. In another aspect, the channel thickness ranges from about 0.05 mm to about 0.5 mm. In another aspect, the residence time of a sample in the chamber ranges from about 5 minutes to about 2 hours. In another aspect, the residence time of a sample in the channel ranges from about 5 minutes to about 1 hours. In another aspect, the channel comprises from about 1 to 8 horizontal/lateral loops and 0 to 8 and vertical/longitudinal loops. In another aspect, the channel comprises one or a plurality of vertical/longitudinal oriented loops. In another aspect, the channel does not comprise any vertical/longitudinal oriented loops. In another aspect, the sample inlet delivers the sample directly into the lower reaction chamber. In another aspect, the sample inlet has a diameter from about 0.5 mm to about 1.5 mm. In another aspect, the sample inlet delivers the sample directly into the upper reaction chamber. In another aspect, the sample inlet has a diameter from about 1.0 mm to about 5.0 mm. In another aspect, the device further comprises an acrylic substrate layer on the same plane as the POEGMA substrate which creates a POEGMA-acrylic border. In another aspect, the device further comprises a bridge feature that allows the sample to cross the POEGMA-acrylic border on the bottom acrylic substrate layer at the edge of the POEGMA substrate without leaking. In another aspect, the device further comprises a tunnel feature built into the adhesive layers and the bottom acrylic substrate layer. In another aspect, the device further comprises a wash buffer delay channel.

Contributors to the embodiments described herein have previously disclosed a microarray assay device described in International Patent Application Publication No. WO 2017/015132 A1, which is incorporated by reference herein for such teachings. The embodiments described herein build on these principles to provide a microfluidic assay device with improved functionality. This device can be referred to as a “vertical D4 microfluidic cassette”, where D4 stands for dispense, dissolve, diffuse, detect.

The embodiments described herein reduce the amount of required manual input, increases user safety, and minimizes the required resources. For example, a user processing an assay needs only to perform a few simple tasks to initiate a test, after which several downstream processes are performed automatically by the cassette, including incubation, removal of unbound sample and reagent, and drying of the imaging region. Additionally, the cassette has an enclosed waste chamber that collects the incubated sample and contaminated wash buffer. After imaging, the entire cassette can be disposed of. This reduces exposure to potentially biohazardous waste. Further, only a few readily available supplies are needed to use the disclosed device. These could include, for example, the sample to be tested, a pipette to add the sample to the cassette, and a dropper of wash buffer.

One embodiment described herein provides a microfluidic assay device, optionally in the form of a layered substrate (or superstrate) that is oriented in a vertical plane. This orientation takes advantage of capillary flow and gravity to advance an analyte through an assay process. The substrate comprises geometric features for the various stages of analysis and a reagent slide.

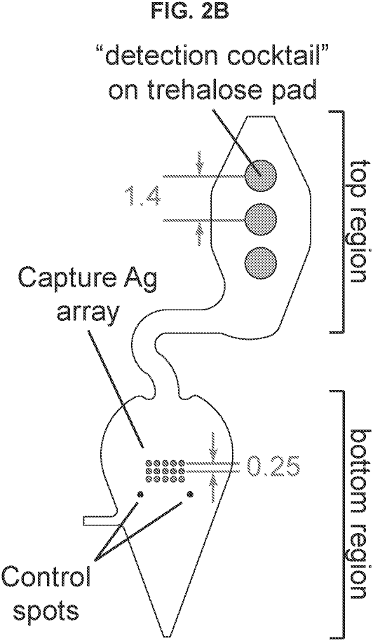

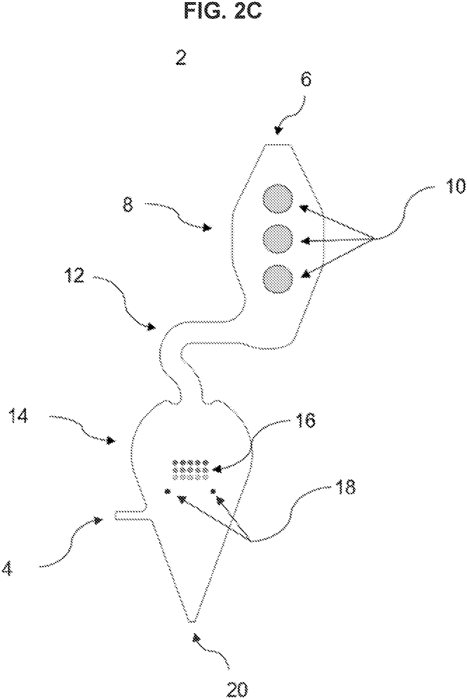

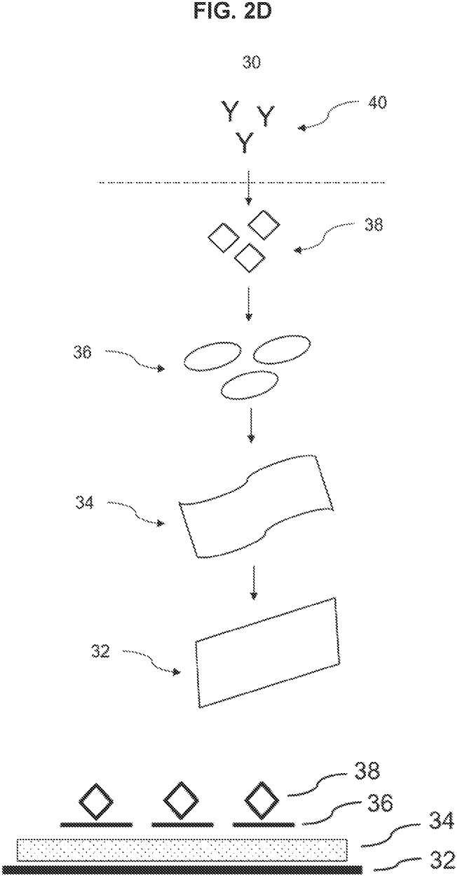

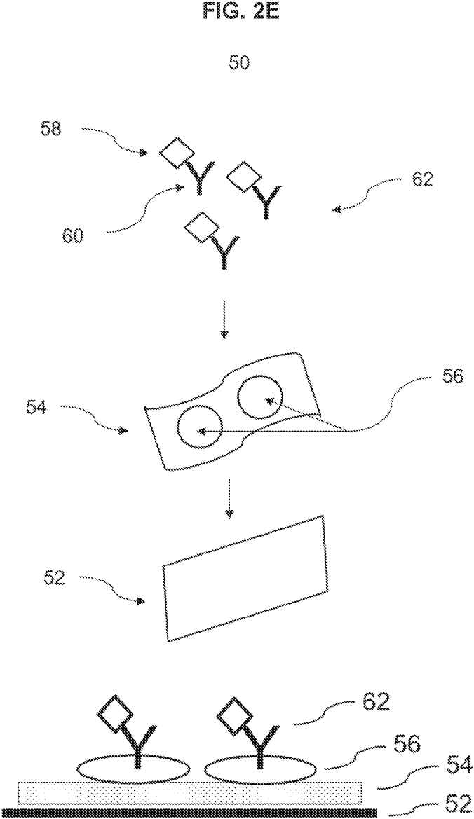

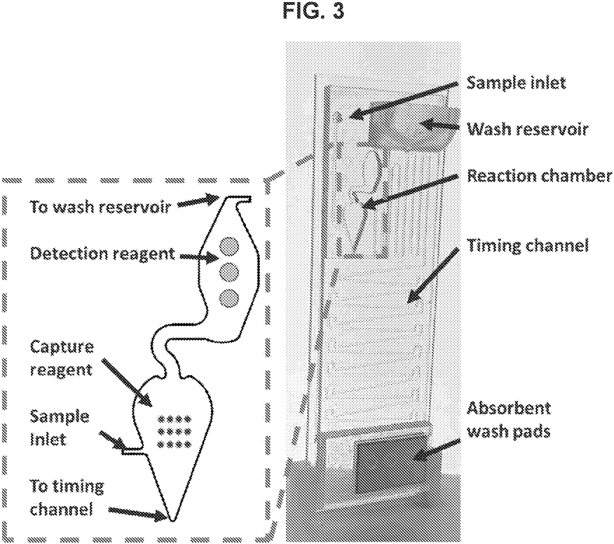

FIG. 3 shows an exemplary embodiment of an assembled cassette. These analysis components and features can be customized to provide optimal analysis for specific analytes. For example, the embodiment shown in FIG. 3 can be used for antigen biomarker detection and double antigen serology assays in human serum, human plasma, or fetal bovine serum. The timing channels can be configured to achieve a particular residence time (e.g., 30 minutes). The example device also includes an optional wash reservoir, which can be attached to or integrated with the cassette.

In general, the timing route using microchannel geometry can promote passive capillary microfluidic flow that drives the assay to completion without the need for actuators or pumps. The vertical alignment of the cassette further assists via introduction of a downward gravitational force that also aids in improving residence time reliability. Additionally, the vertical alignment of the cassette advantageously enables ultra-low background imaging. The POEGMA brush when hydrated does not allow cells or proteins to adhere to its surface. In previous “flat” (e.g., horizontal) versions of the microfluidic cassette, cells could potentially settle onto the imaging surface and could be difficult to remove given the gentle nature of the device’s passive operation. The vertical alignment of the cassette disclosed herein allows cells, debris, and proteins to settle downstream away from the reaction chamber, leaving the imaging area clear.

Further, with the previously disclosed microassay solution, the user is responsible for the addition of the sample, manual timing of the incubation step, removal of sample, addition of wash reagent, removal of wash reagent, and drying of cassette using a benchtop mini centrifuge. The cassette of the present disclosure automates these and other tasks.

Note, it is to be understood that, although the vertical orientation of the cassette provides certain benefits, it can be equally used in other orientations (e.g., oblique, horizontal, or vertical) without departing from the scope of the embodiments described herein.

Additional advantages of the present disclosure include long shelf life, room temperature operation and storage, low volume (e.g., <60 µL) testing. It is capable of detecting multiple biomarkers simultaneously and can be configured for multiple types of assays with minor modifications to the design.

Some non-limiting example analytes include antigen detection (via sandwich fluorescent immunoassay) and antibody detection (via double antigen, and in-direct assay formats). Some non-limiting example input samples include human whole blood, human serum, human plasma, fetal bovine serum, buffered saline solutions, or any other suitable aqueous or liquid input sample.

In some embodiments, the cassette can be configured for direct administration of a sample without a pipette, such as through a finger-prick of blood. In further embodiments, the wash buffer can be optionally integrated into the assembly such that the user can simply activate the flow of wash, rather than adding it manually.

Another embodiment of the present disclosure provides a method of analyzing a sample using the disclosed device. A non-limiting example method is as follows:
1. Remove the cassette from a desiccated pouch.
2. Place the cassette in the provided stand oriented with the sample inlet and reaction chamber at the top (see e.g., FIG. 3).
3. Dispense the sample into the sample inlet of the microfluidic cassette.
4. Dispense the wash buffer into the wash reservoir of the microfluidic cassette.
5. Wait for the assay to run. The following occurs automatically within the microfluidic cassette during the run time:
   Fluorescently labeled reagent dissolves and forms a sandwich with the analyte of interest and the capture reagent inside of the reaction chamber.
   A small volume of sample traverses a snaking timing channel that dictates the residence time of the cassette.
   Once the sample reaches the end of the timing channel it encounters an absorbent material situated at the outlet, which will rapidly absorb the sample inside of the reaction chamber.
   As the sample clears, wash buffer that is situated upstream of the reaction chamber will flow into the reaction chamber before it is itself soaked up by the absorbent material. As the wash buffer flows through the reaction chamber, it collects and removes residual sample and unbound reagent, leaving a clean imaging surface.
6. The cassette is ready for analysis on a conventional or customized scope.

### ADVANTAGES OF THE TECHNOLOGY

### Flow on a POEGMA Substrate

Most microfluidic systems are engineered on PDMS, glass, silicon, or plastics (e.g., PMMA). The disclosed device is engineered on a POEGMA brush substrate. The engineered brush has dynamic properties that change depending on if it is dry or hydrated (i.e., if sample is present or not). Contact angle is one of these properties and is integral in the calculation of capillary flow. As the sample hydrates the brush as it enters the reaction chamber and traverses the timing channels, the contact angle changes in real-time making conventional calculations for capillary flow/pressure difficult to derive. Therefore, optimized designs must be derived empirically, not theoretically.

### Operation in Undiluted Whole Blood

The ability to run undiluted human whole blood with little or no performance loss was a surprising capability of the microfluidic device. Passive capillary quantitative microfluidic devices usually require actuators, pumps, or a filtration step to operate with undiluted human whole blood. It was discovered that it is possible for undiluted whole blood samples of varied hematocrit to flow through the reaction chamber and timing channels of the present device and not become clogged, as long as they were EDTA-treated, by introducing the following unique features: (1) Enclosing the reaction chamber and introducing one-way backflow prevention valves at the inlet to allow for the vertical orientation of the cassette without leakage; and (2) Design of timing channel width, length, and downstream angle to achieve target residence time, but prevent chip failure from blockage of channels with RBCs.

Vertical orientation alone is not enough to overcome cellular fouling from whole blood. This prevented the cellular sedimentation onto the sensing surface due to gravity. Cellular adhesion is also an issue. The non-fouling POEGMA substrate prevents cellular adhesion. So POEGMA together with the vertical orientation prevents cellular sedimentation and adhesion. This enables passive automated incubation, washing, and drying with an undiluted whole blood sample without performance loss. Both are required to achieve the desired performance.

### Extended Residence Times

Extended residence times are difficult in a vertically oriented system. The gravitational forces exert a hydrostatic pressure that speeds up flow as fluid moves downstream from the reaction chamber. This eventually overcomes the decreasing capillary pressure and results in a rapid residence time (e.g., samples traverse the timing channel too quickly) or an excessive loss of sample (e.g., the timing channel needs to be excessively long to achieve desired time). The system must be designed so that timing can be tuned (upwards of 1 hour), but most of the input sample is unexhausted. For example, if a sample is 60 µL, only about 6 µL is consumed (e.g., volume filling dead space and capillary adherence) in the timing channel during incubation.

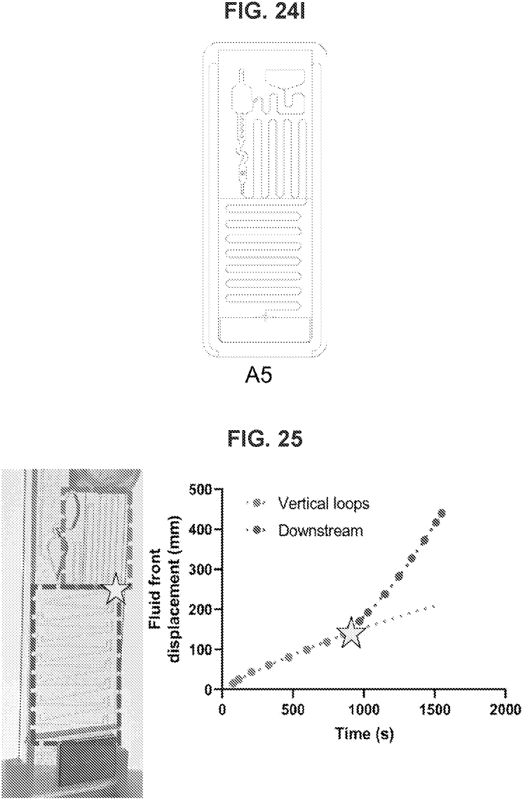

It was discovered that integrating vertical loops in the timing channel which run parallel to the reaction chamber could greatly increase residence time without sample loss. As vertical loops progress upwards parallel to the reaction chamber, the hydrostatic pressure begins to drop. As the fluid front surpasses the meniscus of the sample in the reaction chamber, the hydrostatic pressure goes negative and works against capillary pressure. In this way, precise design of vertical loops can add significant residence/incubation time with minimal exhaustion of sample. This design was critical for non-whole blood samples (serum, plasma, buffer, FBS, urine, etc.). FIG. 25 demonstrates this in practice. The red section of the graph shows residence time in the vertical loops of the channel and demonstrates how the velocity of the fluid front (displacement/time) increases once the fluid front moves past the vertical loops. The length and height of the vertical loops can be tuned to increase or decrease residence time with minimal impact on the amount of consumed sample.

### Recreating D4 Performance

The original D4 assay was purely diffusion driven. Detection reagents passively diffuse towards immobilized capture spots. In a vertically oriented microfluidic system, gravity and the directionality of fluid flow play a role in how detection reagent is delivered to the capture spots. This led to the design and implementation of the following design characteristics: (1) Upstream positioning of detection reagent so that all reagents will dissolve and interact with the capture array, and none will be lost downstream (which occurs if they are printed adjacent or under the capture array); (2) Mixing channel situated between the top and bottom of the reaction chamber to improve the homogeneity of the detection reagent before coming into contact with the capture array (this reduces intra-assay variability; and (3) Offset (part of the mixing channel) between the top and bottom regions of the reaction chamber. A small amount of the printed detection reagent does not fully liberate from the POEGMA brush but can still traverse downstream in the brush. Earlier versions of the cassette without an offset had streaking of the detection reagent through the capture spots, reducing performance. The offset prevents this from happening.

The ongoing severe acute respiratory syndrome-coronavirus 2 (SARS-CoV-2) pandemic poses an enormous challenge to the world. SARS-CoV-2 has resulted in over 47 million cases of coronavirus disease (COVID-19) worldwide, resulting in over 1.2 million deaths as of Nov. 3, 2020. Unlike many other viruses, SARS-CoV-2 displays high infectivity, a large proportion of asymptomatic carriers, and a long residence time of up to 12 days, during which carriers are infectious. As a result, transmission has been widespread, resulting in overwhelmed healthcare capacities across the globe. Timely, reliable, and accurate diagnostic and surveillance tests are necessary to control the current outbreak and to prevent future spikes in transmission. Reverse transcription polymerase chain reaction (RT-PCR), which detects viral nucleic acids, is the current gold standard for COVID-19 diagnosis. Although RT-PCR is highly sensitive and specific, it does not detect past infections-RNA is typically only present at high quantities during acute infection-and it does not provide insight into the host s response to infection. Serological assays, which detect antibodies induced by SARS-CoV-2, are a crucial supplement to nucleic acid testing for COVID-19 management. Specifically, serological assays are important to track the body’s immune response, and to potentially inform prognosis or immunity status. Serological assays are also essential for use in epidemiological studies and are a critical enabling tool for vaccine development.

SARS-CoV-2 is an enveloped RNA virus with four structural proteins: spike (S) protein, membrane (M) protein, enveloped (E) protein, and nucleocapsid (N) protein. As the pandemic unfolded, several serological binding assays were developed including enzyme-linked immunosorbent assays (ELISAs) and lateral flow assays (LFA). These assays measure either the level of total antibody or that of specific antibody isotypes that bind to viral proteins-normally S or N. Several studies have demonstrated promising clinical sensitivity and specificity for ELISA and some LFAs. Furthermore, several ELISAs have been shown to correlate well with neutralizing antibody titers, and thus may be useful clinically and in vaccine development. However, both ELISA and LFAs have major disadvantages that limit their applicability for COVID-19 management. ELISA requires technical expertise, laboratory infrastructure, and multiple incubation and wash steps, limiting its applicability to settings outside of a centralized laboratory. On the other hand, LFAs are portable, but they have lower sensitivity and provide qualitative results, whereas a quantitative readout is preferred for clinical use, research studies, and surveillance applications. Collectively, these shortcomings of ELISAs and LFAs motivate the need for an easily deployable, point-of-care test (POCT) that can be manufactured in large volumes, has quantitative figures of merit equal to laboratory-based tests, and is as easy to use as an LFA.

To address the challenge of creating a user-friendly and widely deployable assay that can detect prior exposure to, and immunological response against SARS-CoV-2, a new multiplexed portable COVID-19 serological assay was developed that is described herein. This passive microfluidic platform provides sensitive and quantitative detection of antibodies against multiple SARS-CoV-2 viral antigens in 60 minutes with a single test from a single 60 µL drop of blood, plasma, or serum. The antibody response against three different SARS-CoV-2 antigens was quantified because emerging studies have demonstrated that the primary antigenic target of the humoral immune response may inform disease progression and prognosis. Thus, being able to differentiate the viral targets of antibodies, as is made possible with the disclosed assay platform, may be especially valuable. Further, the portable test is completely automated and can function independently of a centralized laboratory at the point-of-care. The test is also shown to be easily modified to detect additional protein biomarkers, such as cytokines/chemokines, without compromising the performance of the serological assay, which may provide further clinical insight into disease severity and or patient outcomes. Collectively, these attributes suggest that the disclosed platform is a valuable tool for COVID-19 management both at the individual patient level (i.e., monitoring patients who may progress to severe disease) and for large-scale epidemiological studies at the population level.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A multi-layered microfluidic assay device comprising a cassette comprising a plurality of layers comprising:
a substrate layer comprising a non-fouling polymer layer coated on a glass substrate; and assay reagents disposed upon the substrate layer;
a microfluidic layer comprising a channel and a reaction area in fluid communication with each other and to inlets and outlets, the ultimate microfluidic layer adjacent to a cover layer.

Clause 2. The device of clause 1, wherein the microfluidic layer comprises:
a channel layer comprising a continuous circuitous channel in fluid communication with a reaction layer; and
a reaction layer comprising cutouts for a reaction chamber, an inlet, and an outlet, each in fluid communication with the channel layer, the reaction layer being sandwiched between the channel layer on one side and the cover layer on the other side.

Clause 3. The device of clause 1 or 2, wherein the reaction chamber comprises an upper reaction chamber, a lower reaction chamber, and an offset mixing channel fluidly connecting the upper and lower reaction chambers.

Clause 4. The device of any one of clauses 1-3, wherein the offset mixing channel comprises a P-trap bend to prevent clogging.

Clause 5. The device of any one of clauses 1-4, wherein the assay reagents comprise one or more detection reagents and one or more capture reagents that are spatially separated and disposed on the non-fouling polymer layer to align with the lower and upper reaction chambers.

Clause 6. The device of any one of clauses 1-5, wherein the detection reagents are placed on layers of trehalose, disposed on the substrate spatially at a position corresponding to the upper reaction chamber; and of the capture reagents are disposed on the substrate spatially at a position corresponding to the lower reaction chamber.

Clause 7. The device of any one of clauses 1-6, wherein the cover layer is attached to the reaction layer via an adhesive layer.

Clause 8. The device of any one of clauses 1-7, wherein the channel layer, the reaction layer, and the cover layer are interconnectedly vented to ambient atmospheric pressure.

Clause 9. The device of any one of clauses 1-8, wherein the cover layer comprises one or more inlets and outlets in fluid communication with the microfluidic layers and with a reservoir at the inlets and an absorbent waste pad at the outlets.

Clause 10. The device of any one of clauses 1-9, wherein the non-fouling polymer layer is a poly(oligoethylene glycol methyl ether methacrylate) (POEGMA).

Clause 11. The device any one of clauses 1-10, wherein the channel layer is an adhesive layer or an injection molded plastic layer.

Clause 12. The device of any one of clauses 1-11, wherein the reaction layer is acrylic.

Clause 13. The device of any one of clauses 1-12, wherein the continuous circuitous channel has longitudinal/vertical and lateral/horizontal orientations.

Clause 14. The device of any one of clauses 1-13, wherein the device is configured to operate in a substantially vertical orientation aligned with gravity.

Clause 15. The device of any one of clauses 1-14, wherein the device is configured to operate via gravity-assisted capillary flow for sample transit through the microfluidic layer.

Clause 16. The device of any one of clauses 1-15, wherein the reaction layer has a thickness of 0.2 to 3.0 mm.

Clause 17. The device of any one of clauses 1-16, wherein the reaction chamber length ranges from about 10 mm to about 40 mm.

Clause 18. The device of any one of clauses 1-17, wherein the reaction chamber width ranges from about 2 mm to about 5 mm.

Clause 19. The device of any one of clauses 1-18, wherein the total channel length ranges from about 50 mm to about 600 mm.

Clause 20. The device of any one of clauses 1-19, wherein the channel width ranges from about 0.05 mm to about 2 mm.

Clause 21. The device of any one of clauses 1-20, wherein the channel thickness ranges from about 0.05 mm to about 0.5 mm.

Clause 22. The device of any one of clauses 1-21, wherein the residence time of a sample in the chamber ranges from about 5 minutes to about 2 hours.

Clause 23. The device of any one of clauses 1-22, wherein the residence time of a sample in the channel after it has emptied from the reaction chamber ranges from about 5 minutes to about 1 hours.

Clause 24. The device of any one of clauses 1-23, wherein the channel comprises from about 1 to 8 horizontal/lateral loops and 0 to 8 and vertical/longitudinal loops.

Clause 25. The device of any one of clauses 1-24, wherein the channel comprises one or a plurality of vertical/longitudinal oriented loops.

Clause 26. The device of any one of clauses 1-25, wherein the channel does not comprise any vertical/longitudinal oriented loops.

Clause 27. The device of any one of clauses 1-26, wherein the sample inlet delivers the sample directly into the lower reaction chamber.

Clause 28. The device of any one of clauses 1-27, wherein the sample inlet has a diameter from about 0.5 mm to about 1.5 mm.

Clause 29. The device of any one of clauses 1-28, wherein the sample inlet delivers the sample directly into the upper reaction chamber.

Clause 30. The device of any one of clauses 1-29, wherein the sample inlet has a diameter from about 1.0 mm to about 5.0 mm.

Clause 31. The device of any one of clauses 1-30, further comprising an acrylic substrate layer on the same plane as the POEGMA substrate which creates a POEGMA-acrylic border.

Clause 32. The device of any one of clauses 1-31, further comprising a bridge feature that allows the sample to cross the POEGMA-acrylic border on the bottom acrylic substrate layer at the edge of the POEGMA substrate without leaking.

Clause 33. The device of any one of clauses 1-32, further comprising a tunnel feature built into the adhesive layers and the bottom acrylic substrate layer.

Clause 34. The device of any one of clauses 1-33, further comprising a wash buffer delay channel.

Clause 35. The device of any one of clauses 1-34, wherein the device has functional improvements as compared to conventional devices, including: a longer shelf life; extended incubation times; room temperature storage and operation; low sample volume required for testing; capability of detecting multiple biomarkers simultaneously; and capability of being configured for multiple assay types with minor modifications to design.

Clause 36. Use of the device of any one of clauses 1-35 for analyzing a biological sample by measuring a concentration level of an analyte in the biological sample.

Clause 37. A method for analyzing a biological sample by measuring a concentration level of an analyte, the method comprising:
(a) orienting the device of any one of clauses 1-35 with gravity with the sample inlet at the top;
(b) loading a sample into the sample inlet;
(c) loading a wash buffer into the wash reservoir of the device;
(d) allowing the sample and wash buffer to enter and traverse completely through the device;
(e) imaging the device to measure a signal for the target analytes and controls; and
(f) determining the concentration of the analyte.

Clause 38. The method of clause 37, wherein the sample comprises whole blood, serum, plasma, urine, tears, sweat, saliva, lymph, cerebrospinal fluid, fecal extract, cellular or tissue extracts, or any other aqueous sample.

Clause 39. The method of clause 37 or 38, wherein the analyte is a biomolecule from an infectious agent, cancer, or is a biomarker for cardiovascular disease, or metabolic disorder.

Clause 40. The method of any one of clauses 37-39, wherein the analyte is a biomolecule or biomarker from a host response to an infectious agent, cancer, cardiovascular disease, or metabolic disorder.

Clause 41. The method of any one of clauses 37-40, wherein the analyte is a biomolecule or biomarker for SARS-CoV-2 or Ebola.

Clause 42. The method of any one of clauses 37-41, wherein the analyte is a cancer biomolecule or biomarker.

Clause 43. The method of any one of clauses 37-42, wherein the analyte is a biomolecule or biomarker associated with hepatocellular carcinoma.

Clause 44. A method for fabricating a microfluidic cassette assay device comprising a substrate layer having microfluidic geometry and a reagent layer disposed adjacent to the substrate layer, the method comprising:
(a) depositing a poly(oligoetllylene glycol methyl ether methacrylate) (POEGMA) layer onto a glass substrate;
(b) depositing a trehalose layer upon the POEGMA layer;
(c) depositing a detection reagent onto the trehalose layers and a capture reagent onto the POEGMA layer at sites corresponding to a reaction chamber;
(d) adhering complementary layers of acrylic and adhesive sheets having microfluidic geometries onto the POEGMA substrate coated glass slide base, wherein the microfluidic geometries comprise a sample inlet, a wash reservoir, a reaction chamber comprising an upper chamber and a lower chamber separated by an offset mixing channel, a circuitous channel comprising a plurality of loops, and an outlet; and
(e) attaching a wash reservoir and absorbent waste pad.

Clause 45. The method of clause 44, wherein the acrylic and adhesive sheets are laser-cut to form microfluidic geometries prior to adhering onto the POEGMA substrate.

Clause 46. The method of clause 44 or 45, wherein the acrylic and adhesive sheets are injection molded to form microfluidic geometries prior to adhering onto the POEGMA substrate.

Clause 47. The method of any one of clauses 44-46, wherein the capture reagent and detection reagent are inkjet-printed and spatially separated to align with the corresponding microfluidic geometry of the substrate layer.

Clause 48. The method of any one of clauses 44-47, wherein the capture reagent is printed in the bottom region of the reaction chamber and the detection reagent is printed in the top region of the reaction chamber.

Clause 49. A microfluidic assay system comprising:
the microfluidic assay device any one of clauses 1-35;
a stand;
a wash buffer; and
a sample applicator.

Clause 50. A kit comprising:
the microfluidic assay device of any one of clauses 1-35;
a stand;
a wash buffer; and
a sample applicator.

Clause 51. A multi-layered microfluidic assay device comprising a cassette comprising a plurality of layers comprising:
a substrate layer comprising a non-fouling polymer layer coated on a glass substrate; and assay reagents disposed upon the substrate layer;
a microfluidic layer comprising a channel layer and a reaction layer in fluid communication with each other and to inlets and outlets, the ultimate microfluidic layer adjacent to a cover layer;
   the channel layer comprising a continuous circuitous channel in fluid communication with the reaction layer;
   the reaction layer comprising cutouts for a reaction chamber, an inlet, and an outlet, each in fluid communication with the channel layer, the reaction layer being sandwiched between the channel layer on one side and the cover layer on an opposing side; and
   the reaction chamber comprising an upper reaction chamber, a lower reaction chamber, and an offset mixing channel fluidly connecting the upper and lower reaction chambers.

Clause 52. Use of the device of clause 51 for analyzing a biological sample by measuring a concentration level of an analyte in the biological sample.

### EXAMPLES

### Example 1

### DA-D4 Assay

The DA-D4 assay is based on the design of the D4 immunoassay. Joh et al., Proc. Natl. Acad. Sci. U.S.A 114: E7054-E7062 (2017), which is incorporated by reference herein for such teachings. Briefly, a polymer brush composed of poly(oligoethylene glycol methyl ether methacrylate) (POEGMA) was “grafted from” a glass slide by surface-initiated atom transfer radical polymerization. Recombinant SARS-CoV-2 proteins were then printed onto POEGMA-coated slides as capture and detection spots. Capture spots of the following proteins were printed as ~170 µm diameter spots using a Scienion S11 sciFLEXARRAYER (Scienion AG) inkjet printer: Spike S1 (Sino Biological, cat# 40591-V05H1), Spike RBD (Sino Biological, cat# 40592-VO2H), and Nucleocapsid protein (Leinco, cat# S854). Each protein was printed as a row/column of five identical spots. Next, 12 excipient pads of trehalose with 1.6 mm spacing were printed from a 10% (w/v) trehalose solution in deionized water around the periphery of the capture antigen array using a BioDot AD1520 printer (BioDot, Inc.). To print the detection reagents, S1 (Sino Biological, cat# 40591-V08H) and N-NTD (produced in-house), were first conjugated to Alexa Fluor 647 (per the manufacturer’s instructions) and then detection spots, of the fluorescent protein conjugates of these proteins were printed on top of the excipient pads as twelve 1 mm diameter spots. A schematic of the chip that shows the spatial address and dimensions of the capture spots, trehalose pad and detection spots is shown in FIG. 2A. After printing and final assembly, D4 chips were stored with desiccant until use. The amount of reagent deposited for the open-format and microfluidic format was identical, with the only difference being the relative spot placement (FIGS. 2A-B). For DA-D4 assays that also detected IP-10, an additional column of five spots of capture antibody (R&D systems, cat# MAB266) was included and anti-IP-10 detection antibody (R&D systems, cat# AF-266) was included in the detection cocktail for the open format chips.

### Example 2

### Expression and Purification of N-Terminal Domain of SARS-CoV-2 Nucleocapsid Protein

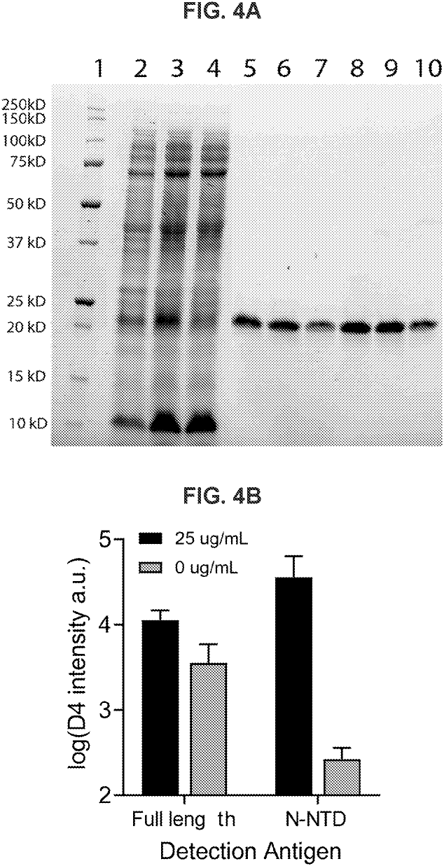

The nucleotide sequence for the N-terminal domain of the nucleocapsid protein (N-NTD) of SARS-CoV-2 (residues 33-212) with a C-terminal His-tag was codon-optimized for E. coli. The gene was synthesized and cloned into a PET-24a vector (Twist Bioscience) and expressed in BL21(DE3) E. coli. Cultures were grown in shaker flasks at 25° C. for 4 h, induced with 0.5 mM IPTG and grown overnight at 16° C. Cells were harvested and lysed by sonication, lysates were clarified by centrifugation, and N-NTD was purified from the lysate supernatant by immobilized metal affinity chromatography (IMAC) (FIG. 4A).

### Example 3

### Fabrication and Analytical Testing of Open Format DA-D4

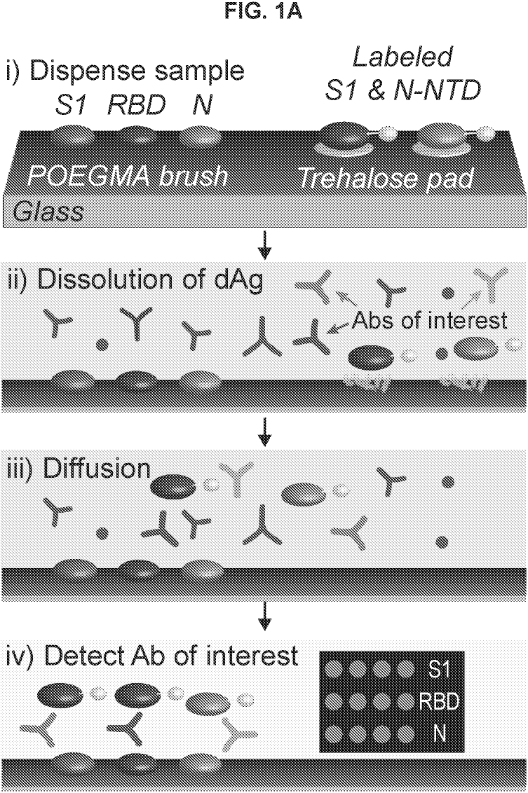

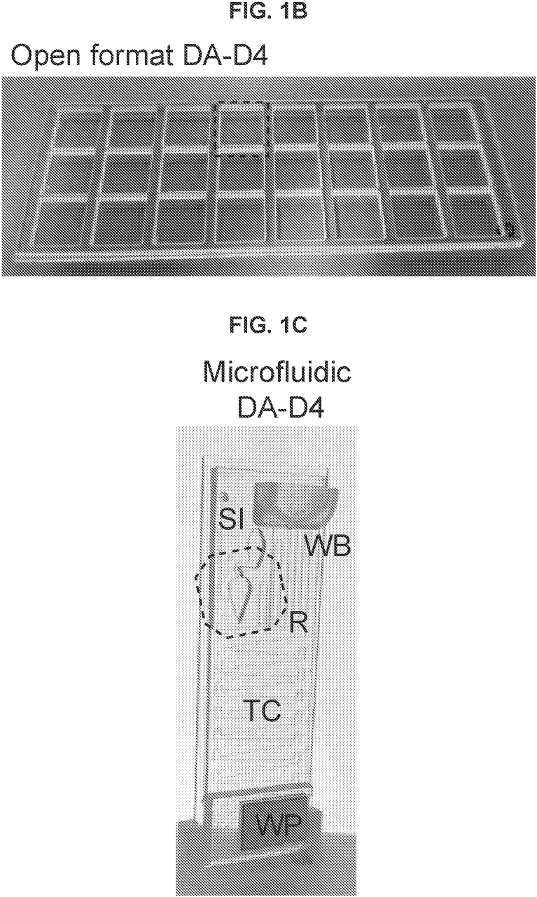

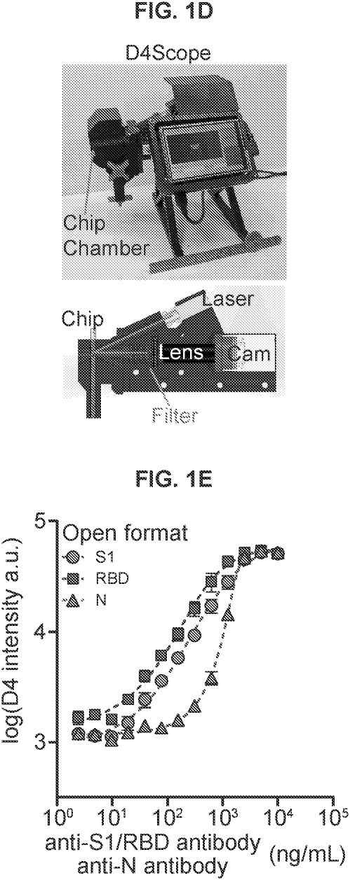

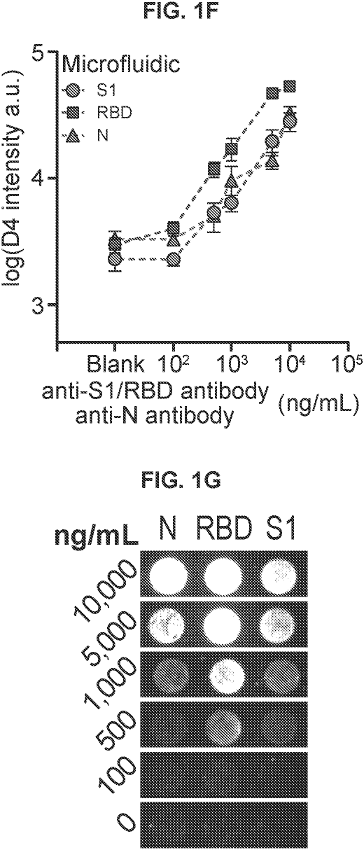

Open format slides were prepared by adhering acrylic wells to each slide, which splits one slide into 24 independent arrays (see FIG. 2A for a schematic and FIG. 1B for an image). To validate the analytical performance of the test, dose-response curves were generated using antibodies targeting SARS-CoV-2 antigens (Sino Biological, cat#: 40143-MM05, 40150-D001, and 40150-0004) spiked into undiluted pooled human serum. Open format chips were incubated with a 15-point dilution series (run in triplicate) for 30 minutes, briefly rinsed in a 0.1% Tween-20/PBS wash buffer and then dried. Arrays were imaged on an Axon Genepix 4400 tabletop scanner (Molecular Devices, LLC).

### Example 4

### Fabrication and Analytical Testing of Microfluidic DA-D4

The microfluidic chip was fabricated by adhering complementary layers of precision laser-cut acrylic and adhesive sheets onto the POEGMA substrate that had been functionalized with the relevant capture and detection reagents. The resulting assembly features a reaction chamber, timing channel, sample inlet, wash buffer reservoir, and wicking pad that automates the sample incubation, sample removal, wash, and drying steps. Simulated doses were prepared using antibodies targeting SARS-CoV-2 antigens (Sino Biological, cat#: 40143-MM05, 40150-D001, and 40150-0004) spiked into undiluted pooled human serum. Six doses (including a blank) were tested on the microfluidic DA-D4 in the following way: (1) The user dispenses 60 µL of sample into the sample inlet using a pipette. (2) The user dispenses 135 µL of wash buffer into the wash reservoir of the cassette using a pipette. (3) The user waits 60 minutes for the cassette to run to completion. During this time, (a) fluorescently labeled antigens dissolve and form sandwiches with the antibodies of interest and the immobilized capture antigen in the reaction chamber. (b) A small volume of sample traverses the timing channel, which governs the residence time. (c) The sample reaches an absorbent pad situated at the end of the timing channel that rapidly wicks away all sample from the reaction chamber, ending incubation. (d) As the sample clears, wash buffer enters the reaction chamber removing residual sample and unbound reagent before it is also wicked away leaving a cleaned and dry imaging surface. Less than a ± 10% variation in the designed 23-minute residence time was observed for the data presented in FIG. 1F. The remaining difference in time accounts for washing and drying time. (4) The cassette is ready for analysis on the D4Scope. The vertical orientation of the cassette works in conjunction with the POEGMA brush to maintain low background fluorescence. Cellular and other sample debris can collect on the brush surface due to gravitational forces, even if no binding is occurring. The vertical orientation ensures that these debris fall harmlessly towards the timing channel during the wash step. This proved especially important when testing with undiluted human whole blood samples.

### Example 5

### Detailed Microfluidic Flow Cell Fabrication

The microfluidic cassettes were fabricated from 1 mm CLAREX acrylic sheets (Astra Products), 9474LE double-sided adhesive tape (3 M company) and Whatman CF7 100 % cotton absorbent liner (Cytiva Life Sciences). 20 DXF files were generated in AutoCAD 2020 (Autodesk, Inc.) and laser-cut using a Gravostyle 8.0 and a LS900 Gravograph CO2 laser (Gravotech, Inc). 3D printed reservoirs and alignment tools were designed in SolidWorks 2019 (Dassault Systèmes SE) and printed on a Form 3 SLA 3D printer (Form Labs, Inc.).

Capture and detection reagents were printed onto the POEGMA coated glass slide in the same way as described in “DA-D4 assay”, with the only difference being the relative placement of the reagents. For the microfluidic flow cell, the detection reagents were printed in the top region of the reaction chamber and the capture spots were printed in the bottom region of the reaction chamber, as shown in FIG. 2B. This placement is a result of the reaction in the flow cell not being purely diffusion-driven, as is the case with the open format. Here, the combination of gravity and a downward direction of fluid flow requires upstream placement of the detection reagent to achieve even and efficient delivery of the detection reagents to the capture spots. The offset nature of the reaction chamber addressed an issue where small amounts of detection reagent that are not completely released from the POEGMA brush travel downstream in the brush during incubation and can lead to a higher background in the imaging area.

The microfluidic fluid cell assembly consisted of five unique stacked layers with two additional attachments (wash reservoir and absorbent pad) shown in the exploded view of FIG. 5A. (i) The fouling-resistant POEGMA coated glass is the base substrate for the flow cell, where reagents are printed, and serves as the back wall of the microfluidic channels. (ii) Adhesive #1 contains the pattern for the 400 µm wide microfluidic channels (timing channel, sample inlet, channel between wash chamber and reaction chamber). The thickness of the adhesive gives the channels a depth of 157 µm. The outline of the reaction chamber and wash reservoir are left exposed to allow the sample and wash buffer to contact the POEGMA substrate. There is an open area at the outlet of the timing channel where the absorbent pad is adhered. The opening is slightly smaller than the pads creating a frame for the pads to be adhered to. (iii) Acrylic #1 provides the front wall of the microfluidic channels enclosing them on all four sides. The reaction chamber is left exposed at this layer to increase the reaction chamber volume to ~60 µL by adding the 1 mm of depth from the acrylic. This ensures that most of the sample remains in the reaction chamber during incubation as some sample traverses the timing channel.

Additionally, this layer has cutouts for the sample inlet, wash reservoir, and absorbent pad that all need to be accessible to either complete assembly or operate the cassette. (iv) Adhesive #2 is a smaller layer that primarily serves as a seal between the two acrylic layers. It has a small crescent shaped flap at the sample inlet to create a one-way valve that prevents backflow after sample addition. The outline of the reaction chamber ensures there is an optically transparent path for the excitation laser to travel through during imaging preventing scattering of laser light on the less transparent adhesive that could impact performance. The outline of the wash reservoir provides access to the wash inlet needed for cassette operation. (v) Acrylic #2 seals the reaction chamber and features the sample inlet and wash reservoir access. (vi) A 3D-printed wash reservoir capable of holding up to 250 µL of wash buffer is attached to the exposed wash inlet (vii) Laser-cut absorbent pads absorb waste from the cassette and is held in place with an acrylic wash cover to reduce exposure of the waste to the end user.

FIG. 5B provides a simplified overview of how the layers of the microfluidic flow cell were fabricated and assembled. (i) Adhesive layers #1 and #2 were fabricated by affixing an adhesive sheet, with its protective liners, to a rigid substrate using a sacrificial layer of double-sided adhesive. This assembly was then laser-cut with the features shown in FIG. 5A. The rigid backing maintains the integrity of the otherwise flimsy microfluidic features. (ii) Acrylic layers #1 and #2 were laser cut separately without any special considerations.

All features were rinsed with a 70% IPA solution and dried with nitrogen gas before assembly. (iii) The protective liners present on the acrylic layers and the top of the adhesive layer were removed. (iv) Acrylic layers #1 and #2 were then affixed onto adhesive layers #1 and #2 respectively. The newly created complementary acrylic/adhesive sub-assemblies can then be easily released from the backing protective liner of the adhesive that is still attached to the rigid backbone (iv) A custom designed 3D-printed positioning tool was then used to align the two sub-assemblies to each other and with the POEGMA substrate. To complete the final assembly, the adhesive backed 3D-printed reservoir was attached at the top of the fluid cell where wash buffer is dispensed. Two absorbent pads are fixed to the outlet of the cassette using exposed adhesive from the Adhesive #1 layer. An adhesive backed acrylic cover was placed over the pads to contain waste. For storage, the assembly is packaged in a thermally sealed pouch with 1 g of silica desiccant and stored at ambient temperature and humidity.

### Example 6

### Patient Samples

De-identified, heat-inactivated EDTA plasma samples (57° C. for 30 minutes) were accessed from the Duke COVID-19 ICU Biorepository; approved by the Duke University Institutional Review Board (IRB). Briefly, eligible patients included in the repository were men and women ages 18 years and above who were admitted to an adult ICU at Duke University Hospital with SARS-CoV-2 infection confirmed by PCR testing and who gave informed consent. Samples were collected on study days 1, 3, 7, 14, and 21. In addition to biological samples, clinical data on these patients were also collected including demographics, laboratory data, and clinical courses.

Ten negative control plasma samples were collected under a normal blood donor protocol and were collected from 2014 to 2019 (prior to the COVID-19 outbreak). All patient information, including demographics, is unknown to the investigator team. An additional 11 negative control samples were purchased commercially (Lee Biosolutions Inc.). Last, 20 negative control samples and 18 samples from patients infected with coronavirus 229E (n = 2), HKU1 (n= 4), NL63 (n = 2), and OC43 (n = 10) were collected. All samples were accessed via an exempted protocol approved by the Duke Health IRB. Blood was either purchased commercially (Innovative Research Inc.) or accessed from the ICU biorepository in EDTA-collection tubes and was tested within 48 hours of sample collection.

### Example 7

### Testing of Patient and Control Samples on the Microfluidic DA-D4

The plasma samples (pre-pandemic healthy controls, specificity panel, and ICU biorepository) were thawed from -80° C. storage and allowed to reach room temperature before testing. Blood samples were tested at room temperature. The same procedure used to test the simulated samples as described in “Fabrication and analytical testing of microfluidic DA-D4” was used for testing of all clinical samples. The only exception was that a modified microfluidic flow cell that required the use of 200 µL of wash buffer was used for testing whole blood.

### Example 8

### Microfluidic Flow Cell Operation With Whole Blood

To test whole human blood from EDTA-collection tubes, three modifications were made to the microfluidic flow cell (FIG. 10). First, the timing channel was shortened, and the large vertical loops were removed. The high viscosity of blood and tendency for red blood cells (RBCs) to pack near the capillary fluid front reduces flow rate and increases the likelihood of clogging when the direction of flow is against gravity. This change resulted in a reliable residence time that is comparable to that observed with the plasma flow cell.

Second, a 30° slope was added to the offset channel that separates the top region and bottom region of the reaction chamber. The original design, with no slope, collected RBCs that settled during incubation and were not removed during the wash step. These RBCs would only be flushed into the bottom region of the reaction chamber during the final drying step. This resulted in an undesirable increase in fluorescence background and decreased performance metrics of the assay. The 30° slope prevents this settling from occurring.

Third, the timing channel outlet, which interfaces with the absorbent pad, was modified to prevent blockage of the channel by densely packed RBCs at the capillary fluid front. In the original design, when this dense cell layer reached the outlet-absorbent pad interface, wicking would not commence due to a lack of plasma at the interface. By approaching the wicking pad from the side and adding an 80° slope to the outlet-wicking pad interface, the RBC dense fluid front can flow down the slope allowing more plasma-rich sample to reach the wicking pad interface and activate wicking.

### Example 9

### D4Scope Fabrication and Operation

The D4Scope’s optical elements - the laser, bandpass filter, lens, and camera - and processing elements - the Raspberry Pi 4, touchscreen, and cabling - are mounted in a custom 3D printed chassis. Fully assembled, it weighs ~5 pounds. The D4Scope can be powered either through a portable battery pack or wall power. Once connected to the power source, the D4Scope automatically runs a custom imaging Python program. The user removes the light protection cover from the cassette loading port and slides the microfluidic cassette with glass side towards the detector. The light protection cover is then replaced enclosing the cassette. The user is then prompted to enter the sample ID # and chip I D # using either the touchscreen or optional attached keyboard and mouse.

The D4Scope has two fine adjustment knobs on the cassette loading port that allow for precise vertical and horizontal movement of the cassette relative to the laser source to ensure that the DA-D4 array is perfectly centered with the excitation source. Each array has co-printed two control spots that will always be uniformly bright across all tested samples and align with two super-imposed alignment cross hairs on the live video-feed of the D4Scope. Using the “toggle video” function on the user interface activates the laser and camera to provide a live view of the imaging area for this alignment. Once aligned, the “toggle video” function can be pressed again to end the live view, and the “capture image” function can be used to collect and save the resulting image onto the on-board hard-drive and, optionally, to a cloud-based server defined by the end-user. The live-view feature should be used sparingly to prevent photo-bleaching of the sample. For this study, the resulting fluorescence intensity was manually analyzed using Genepix Analysis software. However, an algorithm has been developed for automatic analysis of spot intensity and instantaneous results on the open-format platform.

### Example 10

### Detailed D4Scope Fabrication

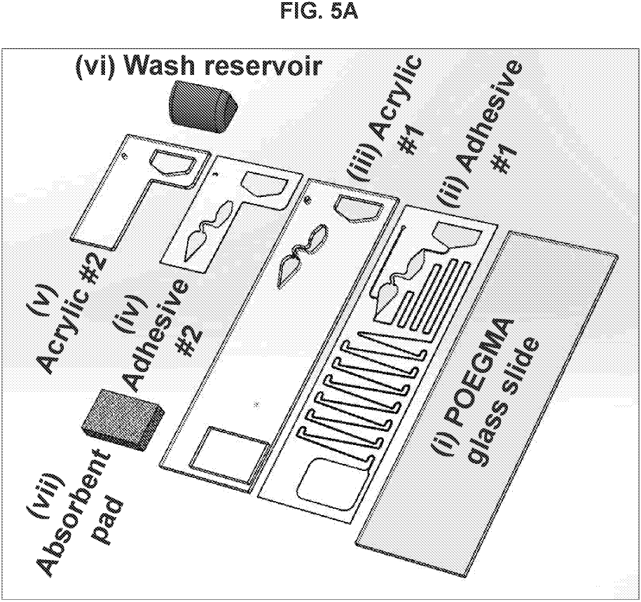

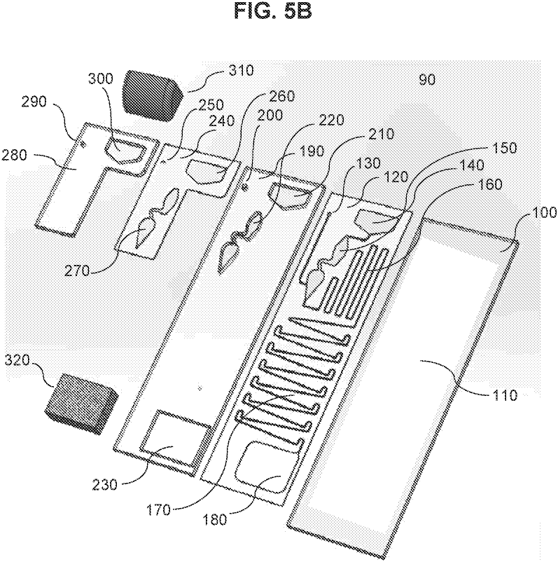

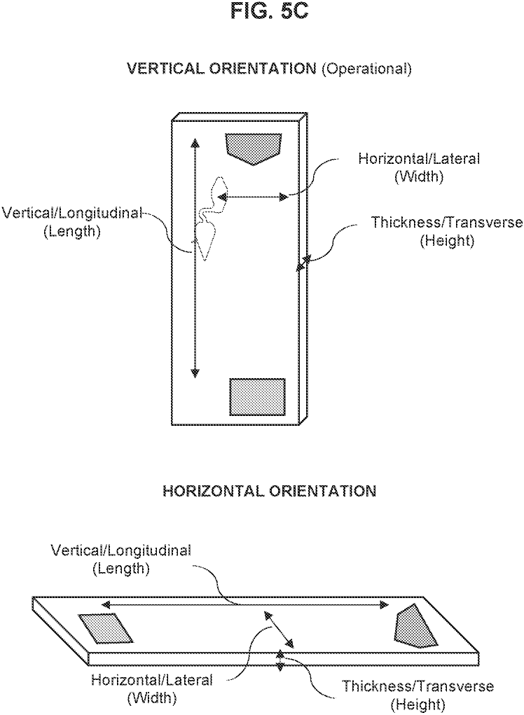

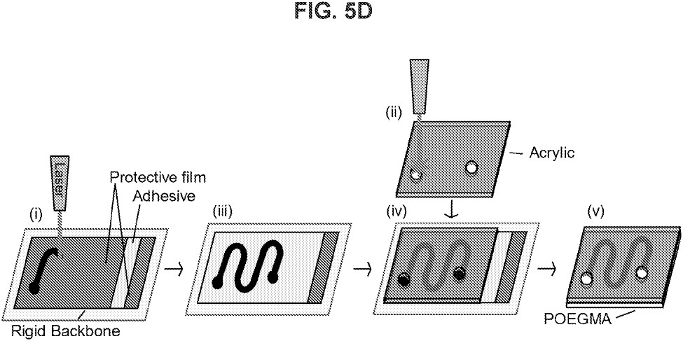

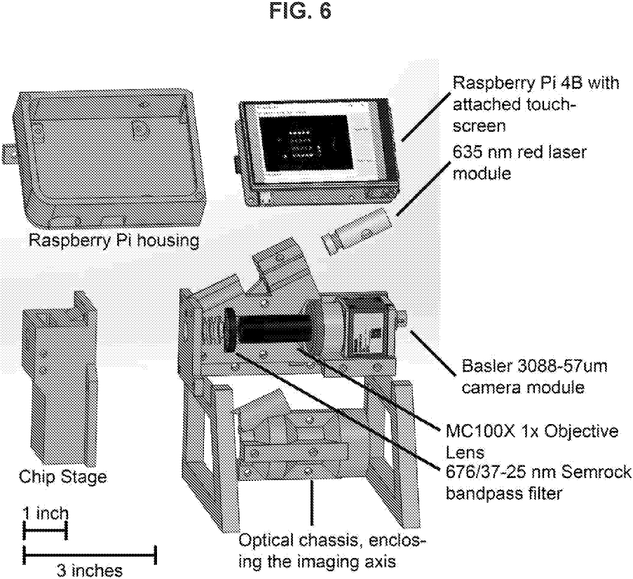

The D4Scope was constructed using a Basler Ace CMOS Camera module AcA3088-57 µm (Basler AG), 676/37-25 nm bandpass filter (Semrock), MC100X lens (Optoengineering), 185 mW 638 nm red laser module (Sharp), Raspberry Pi 4B 2GB (Raspberry Pi Foundation), 3.5 inch TFT LCD display (UCTRONICS), and custom 3D printed housing parts made of polylactic acid filament (HATCHBOX PLA). The D4Scope is designed using SolidWorks (Dassault Systemes SE) in assembly mode. The exploded view is shown in FIG. 6. First, the optical components were positioned by mates to mount the objective lens at the listed working distance (47 mm) away from the D4 chip, the optical filter in-line and concentric with the lens, and the red laser obliquely angled 30° from the imaging axis. Then, the housing parts were designed around these optical components. Special considerations were made to ensure parts are (1) modular for quick prototyping, (2) 3D printable with minimal supports and post-processing, and (3) reasonably assembled. The 3D printed parts are printed on a CR-10 Mini (Creality) and Taz 6 {(Lulzbot) fused deposition modeling 3D printers.

### Example 11

### Live SARS-CoV-2 Microneutralization Assay (MN)

The SARS-CoV-2 virus (Isolate SA-WA1/2020, NR-52281) was deposited by the Centers for Disease Control and Prevention and obtained through BEl Resources, NIAID, NIH. SARS-CoV-2 Micro-neutralization (MN) assays were adapted from a previous study. In short, plasma samples are diluted two-fold and incubated with 100 TCID50 virus for 1 h. These dilutions are transferred to a 96 well plate containing 2×104 Vero E6 cells per well. Following a 96-h incubation, cells were fixed with 10% formalin and CPE was determined after staining with 0. 1% crystal violet. Each batch of MN includes a known neutralizing control antibody (Clone D001; SINO, CAT# 40150-D001). Data are reported as the inverse of the last dilution of plasma that protected from CPE, log₁₀ transformed.

### Example 12

### IP-10 Experiments

Open format DA-D4 slides were fabricated as described above using all reagents needed for antibody detection and IP-10 detection. Citrated plasma samples from 10 patients were procured from the ICU biorepository. 60 µL of each sample was added to two separate DA-D4 chips, incubated for 30 min, and the chips were then rinsed using 0.1% Tween in 1x PBS. All slides were scanned with the Genepix tabletop scanner.

IP-10 levels were measured using the LEGENDplex™ Human Proinflammatory Chemokine Panel (13-plex) and LEGENDplex™ Human Anti-Virus Response Panel (13-plex) obtained from BioLegend. Assays were performed with patient serum per the manufacturer’s instructions. The assay was performed using a Beckman Coulter CytoFLEX flow cytometer and data processing was performed using BioLegend s Bio-Bits cloud-based software platform. Each sample was tested in triplicate, and the results are reported as mean of these triplicates.

### Example 13

### Multiplexed D4 Assay for COVID-19 Prognosis

Multiplexed assays were fabricated as previously described. Capture antibodies (cAbs) for IL-6 (cat#: AF-206, R&D Systems), NT-proBNP (cat #: MAB-9538, R&D Systems), and D-dimer (cat#: ABS-015-22-02, Invitrogen) were adjusted to 1 mg/mL and then printed onto POEGMA-coated slides using a Scienion S11 sciFlexarrayer as separate rows of 350 pL spots. Detection antibodies (dAbs) for IL-6 (cat #: MAB-206, R&D Systems), NT-proBNP (cat#: 267698C, Abcam), and D-dimer (cat#: MAB-104712, R&D Systems) were labeled with AlexaFluor 647 per the manufacturer instructions. Next, twelve excipient pads of trehalose of ~1 mm diameter with 1.6 mm spacing were printed from a 10% (w/v) trehalose solution in deionized water around the periphery of the cAb array using a BioDot AD1520 printer (BioDot Inc.). Then, each dAb was adjusted to 0.02 mg/mL and one 100 nL drop of ~1 mm in diameter was printed upon each trehalose pad (12 total drops) using a BioDot AD1520. After printing, chips were placed in a vacuum desiccator (30 kPa) overnight.

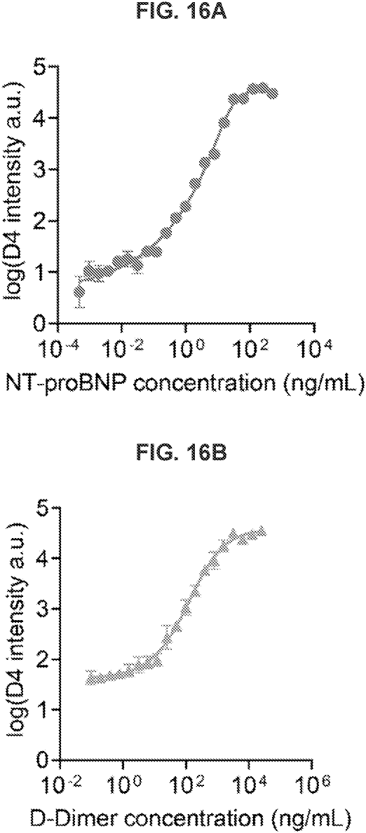

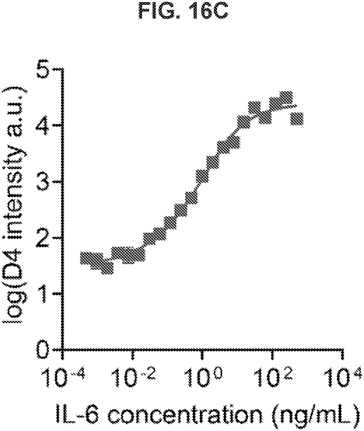

Dose-response curves were generated by incubating D4 chips with a dilution series of IL-6, D-dimer, and NT-proBNP spiked fetal bovine serum (FBS) for one hour on a nutating rocker (FIGS. 16A-C). Afterwards, chips were submerged and washed in 0.1% Tween-20/PBS wash buffer, then in deionized water. Chips were then dried by brief centrifugation. Finally, chips were imaged on an Axon Genepix 4400 tabletop scanner (Molecular Devices, LLC), and analyzed using the GenePix software.

### Example 14

### Statistical Analyses

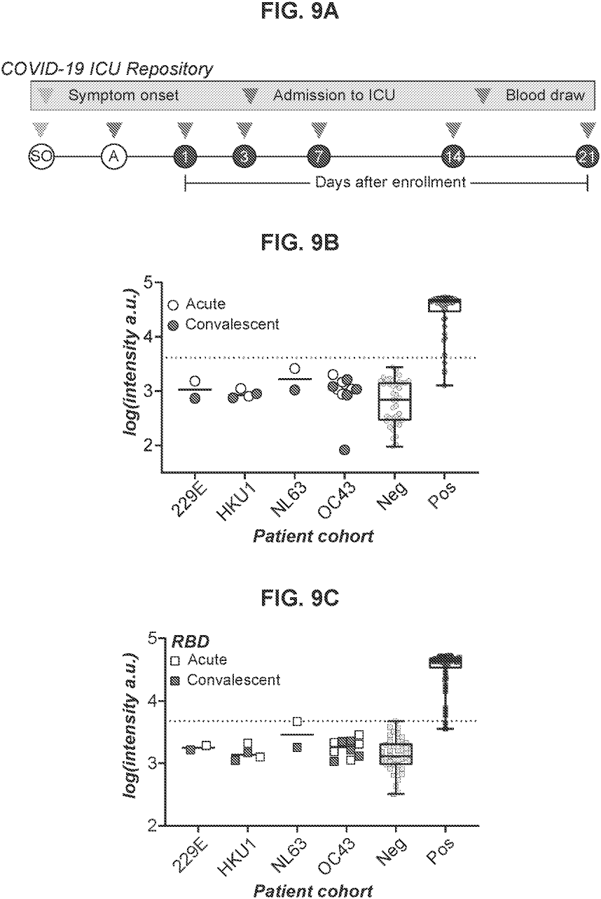

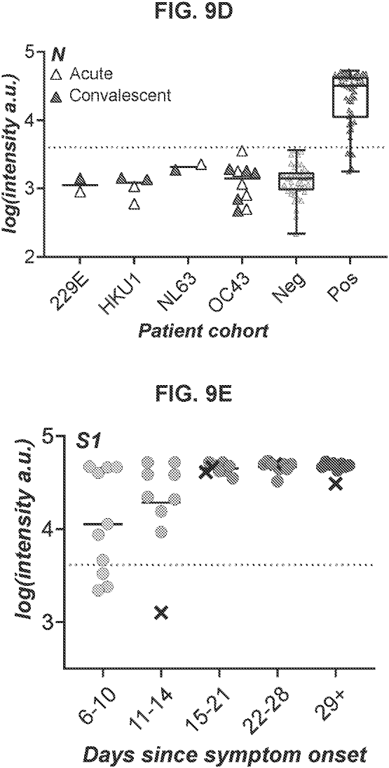

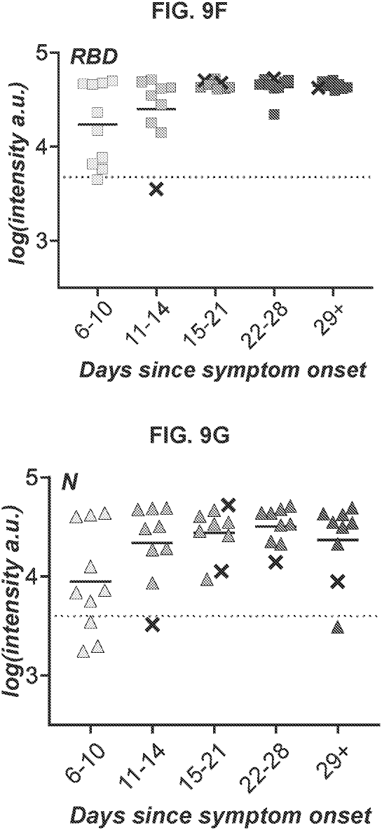

Statistical analyses were performed using GraphPad Prism version 8.4.1 (GraphPad Software, Inc). All data were log-transformed for analysis. To establish statistical significance between negative and positive cohorts (FIGS. 9B-D), unpaired t-tests were used. When comparing multiple groups, a one-way ANOVA followed by Tukey’s post-hoc multiple comparisons test was used. Pearson r correlation was used to assess the degree of correlation between measurements.

### Example 15

### The DA-D4 Point-Of-Care Test (POCT) for COVID-19 Serology

The strategy to evaluate the antibody response to SARS-CoV-2 was based on the D4 assay platform. The D4 platform is a completely self-contained immunoassay platform fabricated upon a “non-fouling” poly(oligoethylene glycol methyl ether methacrylate) (POEGMA) brush, where all reagents needed to complete the assay are inkjet-printed directly onto the surface. In previous work, this platform has been used for the detection of several protein biomarkers using a fluorescent sandwich immunoassay format. Here, the design of the assay was modified to detect antibodies against SARS-CoV-2 using a double-antigen (DA) bridging immunoassay format, which detects total antibody (all isotypes and subclasses). The DA-D4 is fabricated by inkjet printing viral antigens as stable and spatially discrete capture spots. In addition, viral antigens are labeled with a fluorescent tag and are printed nearby on an excipient pad as dissolvable spots.

When a sample was added to the assay (FIG. 1A(i)), the excipient pad dissolves and liberates the fluorescently labeled antigen (FIG. 1A(ii)), which then diffuses across the polymer brush to the capture spots and labels any antibody that has been captured from solution by the stable capture spots of unlabeled antigen (FIG. 1A(iii)). The fluorescence intensity of the capture spots is then imaged using a fluorescent detector and scales with antibody concentration in a sample (FIG. 1A(iv)). Because capture spots of each antigen are printed at spatially discrete locations, this design enables multiplexed quantification of multiple target antibodies using a single fluorescent tag, which greatly simplifies the detector design and assay readout.

To fabricate a serological assay for SARS-CoV-2, nucleocapsid (N), spike S1 domain (S1), and the receptor-binding domain (RBD) of S1 were inkjet-printed as the “stable” capture reagents onto POEGMA-coated slides. The rationale for simultaneously assaying the antibody response towards N, S1, and RBD antigens is that it is not fully understood which epitopes elicit an immune response in all individuals, although they are all believed to be immunogenic and studies have shown that the primary target of the immune response may inform disease prognosis. N is expressed abundantly by SARS-CoV-2 during infection and is highly immunogenic in other coronaviruses. The S protein, composed of the S1 and S2 domains, is exposed on the viral coat of SARS-CoV-2 and plays an essential role in viral attachment, fusion, entry, and transmission. Because S2 is highly conserved across many coronaviruses and is thus potentially cross-reactive, S1 was chosen for antibody detection. RBD - the portion of S1 that binds cells expressing viral receptor - is the target for many neutralizing antibodies and is thus a promising antigenic target for serological assays. FIG. 2A shows the layout and dimensions of an open format DA-D4 chip. Each chip contains 24 individual assays with S1, RBD, and N antigens arrayed as separate rows of five identical ~170 µm diameter spots. Next, fluorescent conjugates of S1-which contains the amino acid sequence for RBD- and the N-terminal domain (NTD) of N (produced in-house, see FIG. 4A for SDS-PAGE of expression and purification) were mixed 1:1 and inkjet-printed as twelve identical 1 mm diameter spots on an identically sized trehalose pad (FIG. 2A). N-NTD, instead of full-length N, was chosen as the detection reagent because the full N domain can dimerize in solution, potentially leading to a false positive result in the DA format.

### Example 16

### Analytical Validation of the DA-D4 POCT Using Simulated Samples

The DA-D4 assay was first demonstrated to detect antibodies against recombinantly expressed SARS-CoV-2 antigens. Initially, the analytical performance was characterized using the open format DA-D4 (FIG. 1B). This is because the open format DA-D4 assay has been extensively optimized and characterized and has extremely high analytical sensitivity, which enables the determination of the figures-of-merit that are theoretically possible for a particular D4 assay. A disadvantage of the open format DA- D4 assay, however, is that it requires a rinse step by the user after incubation of the sample.

For point-of-care deployment and an improved user experience, a new gravity and capillary driven “passive” microfluidic flow cell was developed that fully automates the assay (FIG. 1C). The microfluidic flow cell is fabricated by adhering complementary layers of precision laser-cut acrylic and adhesive sheets onto the functionalized POEGMA substrate (see FIG. 5A and FIG. 2B for the print layout). The resulting microfluidic flow cell features a reaction chamber, timing channel, sample inlet, wash buffer reservoir, and wicking pad that automates the sample incubation, sample removal, wash, and drying steps. This simplifies the user experience and limits the possibility of a user incorrectly carrying out the test, as it only requires the user to add the sample and a drop of wash buffer to the cassette. After ~60 minutes, the cassette is ready for imaging with a custom-built fluorescent detector-the D4Scope (FIG. 1D).

The D4Scope is a low-cost (<$1,000), portable fluorescence detector (with dimensions of 7 inches wide, 6 inches tall, 5 inches deep and a weight of ~5 pounds; see FIG. 6 for dimensions and image) built from off- the-shelf components and assembled using 3D-printed parts that can image microarray spots with high sensitivity. It utilizes coherent 638 nm red laser light set at an oblique angle (30°) relative to the surface to excite the fluorescently labeled antigens. The fluorescence wavelength emission from the labeled reagents is then sent through a bandpass filter and imaged with a high-efficiency Sony IMX CMOS sensor in a Basler Ace camera (FIG. 1D). This setup provides a large field-of-view of 7.4 mm × 5 mm and a fine (raw) lateral resolution of ~2.4 µm. A user-friendly interface was developed in Python that runs on a 3.5-inch Raspberry Pi touchscreen to control laser excitation, camera exposure, and image file output (see FIG. 6).

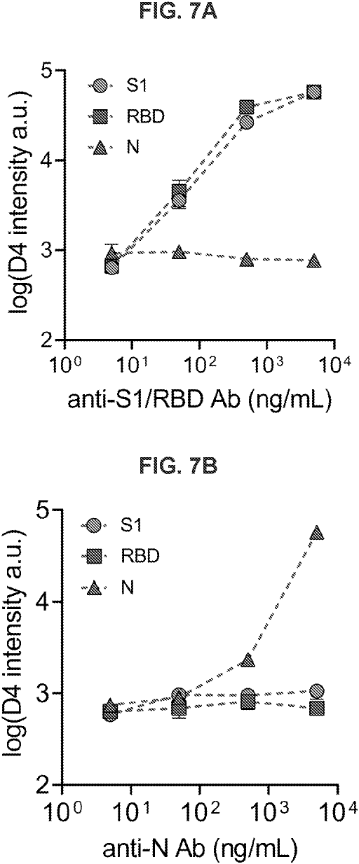

To mimic seropositive samples, commercially available antibodies (with known binding affinity towards SARS-CoV-2 antigens) were spiked into undiluted pooled human serum that was collected prior to the COVID-19 outbreak. A dilution series spanning four logs was evaluated on open format DA-D4 chips and yielded a dose- response curve with fluorescence intensities that scaled with antibody concentration and approximated a sigmoidal curve, demonstrating that the assay was responsive to the antibodies of interest (FIG. 1E). Within the microfluidic flow cell, the chamber geometry, reagent spacing/alignment, and amount of printed reagent were iteratively optimized to match the performance metrics of the open format DA-D4. Six doses (including a blank) with varying amounts of anti-S1/RBD and anti-N antibodies were prepared and tested in quadruplicate on 24 separate microfluidic flow cells to demonstrate equivalence between the open format (FIG. 1B) and microfluidic flow cell (FIG. 1C). In the microfluidic flow cell, the fluorescence intensity of the capture antigens, imaged with the D4Scope, also scaled with antibody concentration, suggesting that the test is responsive to anti-SARS- CoV-2 antibodies (FIGS. 1F-G). Anti-S1/RBD antibodies did not cross-react with N antigen, and anti-N antibodies did not react with S1 or RBD antigens (FIGS. 7A-B).

### Example 17

### Clinical Validation of the DA-D4 POCT

The clinical performance of the DA-D4 POCT in a retrospective study was validated using banked plasma samples from patients with PCR-confirmed COVID-19 who had been admitted to the intensive care unit (ICU) at Duke University Medical Center. A total of 34 COVID-19 positive plasma samples (heat-inactivated) from 19 patients, some of which had longitudinal samples available, and 10 negative samples (collected prior to the COVID-19 pandemic) were tested on the microfluidic DA-D4 and imaged with the D4Scope. The median age of the COVID-19 patients was 55. Of the 19 patients, 10 were female and 9 were male. For most patients, the date of symptom onset was known (29 out of 34 samples), where the average was 20.7 days with a range of 6-48 days. The complete patient profile is provided in Table 1.

TABLE 1

COVID-19 patient clinical characteristics (FIGS. 9 )

| Characteristic | Patients (N = 31) |
|---|---|
| Median age (range) | 55 (36-78) |
| Male sex | 21 |
| Race/ethnicity | |
| Black | 13 |
| Latino | 12 |
| White | 5 |
| Other | 1 |
| Coexisting conditions | |
| Any coexisting condition | 26 |
| Diabetes | 15 |
| Obesity | 15 |
| Hypertension | 15 |
| Cardiovascular disease | 6 |
| Renal Disease | 3 |
| Chronic Lung Disease | 6 |
| Stroke | 4 |

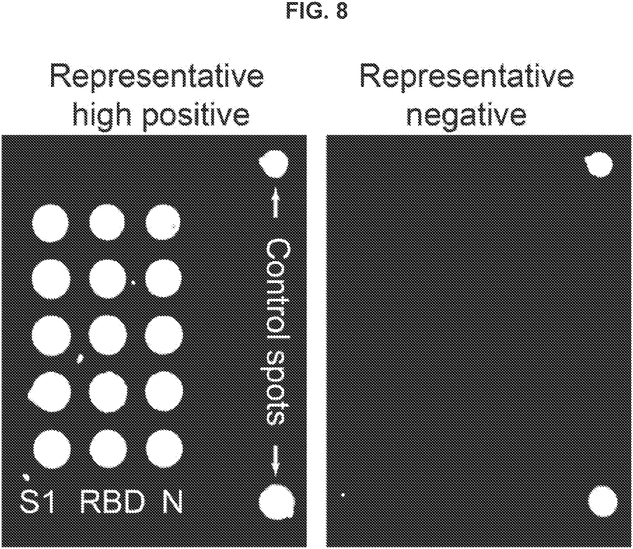

Antibody reactivity towards all three viral antigens was measured on a single microfluidic flow cell for each patient sample. For validation, the threshold for a positive test result was assigned as two standard deviations above the mean of the 41 pre-pandemic negative control samples, which was calculated individually for S1, RBD, and N. There was a statistically significant difference between the mean intensity for COVID-19 positive and negative samples (p < 0.0001) for all three markers (FIGS. 9B-D). Furthermore, all 41 healthy negative control samples tested below the threshold for each marker (specificity of 100%) and all samples within the specificity panel of similar coronavirus infections-both acute and convalescent-also tested below the threshold (specificity of 100%), indicating that the test is highly specific to SARS-CoV-2 (Table 2). Representative images for a high positive and negative sample are included in FIG. 8.

TABLE 2

Specificity and Sensitivity of the DA-D4
Specificity

| Cohort | S1 | RBD | N |
|---|---|---|---|
| Healthy | 41/41 | 41/41 | 41/41 |
| Coronavirus 229E | 2/2 | 2/2 | 2/2 |
| Coronavirus HKU1 | 4/4 | 4/4 | 4/4 |
| Coronavirus NL63 | 2/2 | 2/2 | 2/2 |
| Coronavirus OC43 | 10/10 | 10/10 | 10/10 |
| Sensitivity | | | |
| Days since symptom onset | S1 | RBD | N |
| 6-14 | 15/19 = 78.9% | 17/19 = 89.5% | 15/19 = 78.9% |
| 15+ | 27/27 = 100% | 27/27 = 100% | 26/27 = 96.3% |

Next, the data were partitioned into five different groups based on days since symptom onset: 6-10 days, 11-14 days, 15-21 days, 22-28 days, and > 29 days (FIGS. 9E-G). For two patients (five total samples), the date of symptom onset was unknown, hence the days since first positive RT-PCR test result were used instead (these data points are marked with an X). The sensitivities obtained for S1, RBD, and N at various time frames are summarized in Table 2. For antibodies targeting S1 and RBD, the sensitivity reaches 100% 2 weeks after symptom onset. These results suggest that the assay spans a useful temporal range to detect the dynamic production of antibodies that typically occurs within 2 weeks of symptom onset. In addition, all tested patients developed a robust and sustained antibody response against S1 and RBD.

For antibodies targeting N, there was also a statistically significant difference in DA-D4 readout between groups as determined by one-way ANOVA (p < 0.05). However, the production of N-targeting antibodies appears to occur later, as there was no statistically significant difference in the antibody response when comparing days 6-10 and 11-14 (p > 0.05), but all groups after 15 days were significantly higher than the first time point (p < 0.05). The concentration of N-targeting antibodies also appears to be more variable across all patients, especially at later time points, with some samples testing close to the threshold value. This could be due to the fact that some patients may develop a stronger response against other viral antigens/epitopes (RBD or S1) or against an epitope of N not within the NTD, highlighting the importance of testing for antibodies against several antigens simultaneously to maximize test sensitivity and specificity.

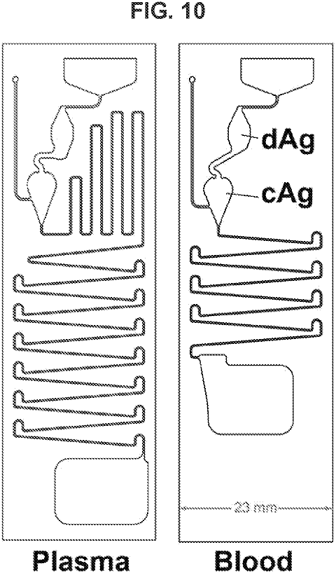

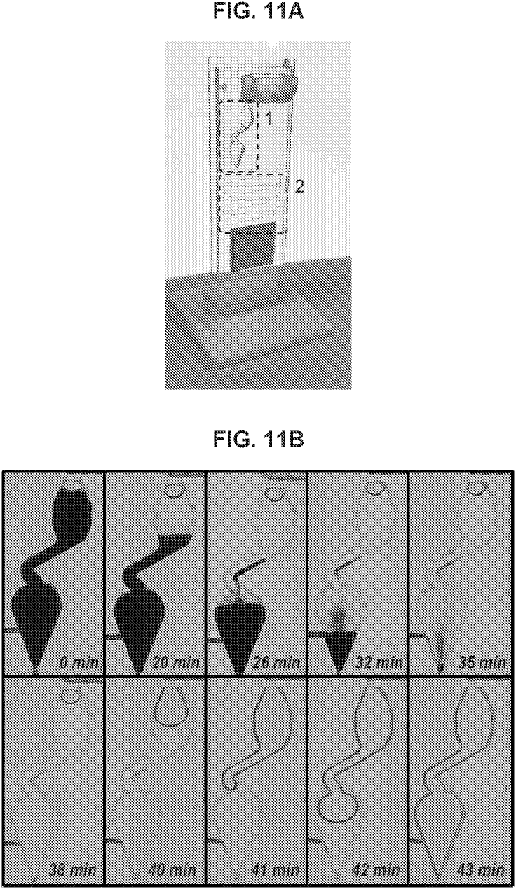

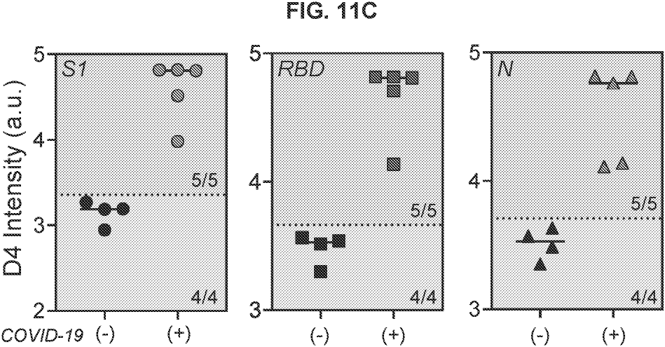

A proof-of-concept study was also conducted using whole human blood as the sample source for the microfluidic flow cell, to demonstrate that the DA-D4 assay can be used at the point-of-care or the point-of-sample collection without the need for any sample processing. To do so, minor modifications, as shown in FIG. 10, were made to the microfluidic timing channel and reaction chamber to account for the non-Newtonian fluid mechanics of whole blood (FIGS. 11A-B). Briefly, a gradual slope was added to the reaction chamber to prevent accumulation of red blood cells during washing, and the incubation channel was shortened to account for a reduced flow rate.

Fresh blood was collected in EDTA-coated tubes from four patients with negative COVID-19 antibody status (as determined by ELISA performed by the supplier) and from five patients with confirmed COVID-19 (from new enrollments to the ICU study) (Table 3). Each 60 µL blood sample was tested on the microfluidic DA-D4 assay. No complications were observed, such as coagulation of blood that can occur when testing whole blood in microfluidic systems. FIG. 11B shows representative images of the reaction chamber, with the time since sample addition noted in the lower right-hand corner of each sub-panel, demonstrating the ability of the microfluidic chip to process whole blood. The antibody response towards S1, RBD, and N from whole blood is shown in FIG. 11C. The threshold to determine a positive test result was set as three standard deviations above the mean of the negatives. All negative samples tested as negative, and all positives tested above the threshold. These preliminary results suggest that the microfluidic DAD4 assay is capable of detecting anti-SARS-CoV-2 antibodies in whole blood, so that the assay can be carried out immediately at the point of sample collection without the need for transport to a centralized laboratory for sample processing into serum or plasma and subsequent testing.

TABLE 3

Clinical characteristics of COVID-19 patients in blood testing study (FIGS. 11 )

| Characteristic | Patients (N = 5) |
|---|---|
| Median age (range) | 56 (26-76) |
| Male sex | 4 |
| Race/ethnicity | |
| Black | 1 |
| Latino | 0 |
| White | 3 |
| Other | 1 |

### Example 18

### Monitoring Antibody Levels Longitudinally

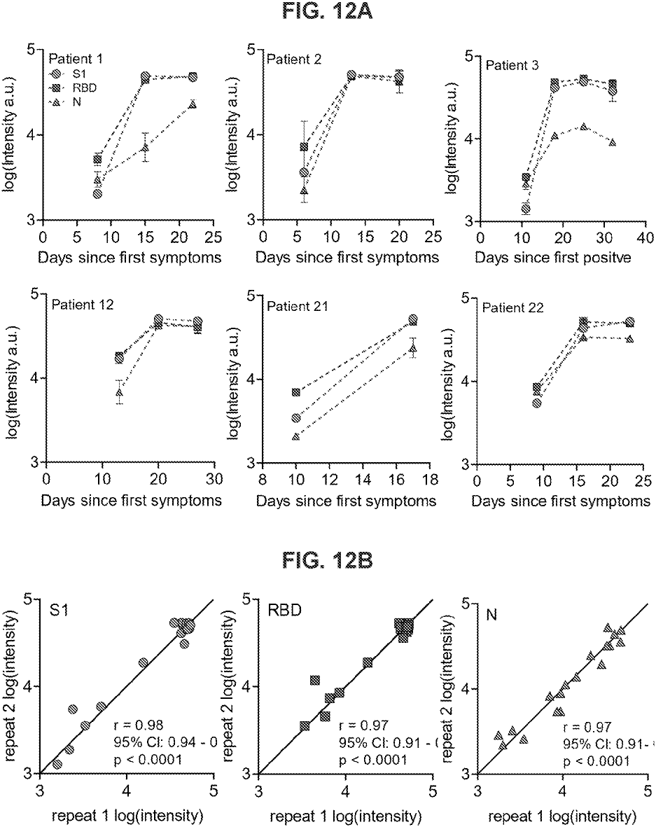

Having demonstrated the high clinical sensitivity and specificity of the microfluidic DA-D4 assay for detection of antibodies against SARS-CoV-2 antigens, as well as the ability to detect changes in antibody levels with time, individual patients were tracked to monitor their seroconversion. To accomplish this, longitudinal plasma samples from six individual patients were tested (FIG. 12A).

Across all six patients, the antibody response was initially low for the first time point tested and then increased and plateaued at later time points, consistent with the antibody dynamics reported in other studies. The DA-D4 readout for antibodies targeting S1 and RBD appeared to saturate by the second time point-typically 2-3 weeks post symptom onset-suggesting that each patient mounted a strong and robust immune response that was sustained over time. For N, the dynamics were slower in one patient (#1) and did not fully saturate in another (#3), providing insight into the primary target of the antibody response in those patients. In general, patients with severe COVID-19 often develop very high antibody titers, which is reflected in this ICU patient sample set by saturated signals at later time points. However, seroconversion was still able to be measured and antibody kinetics in each patient, suggesting that the DA-D4 is a useful tool for monitoring the immune response. The earliest time points for each patient were also still elevated relative to the negative controls, indicating that seroconversion may have been able to be detected earlier, had samples from earlier time points been available. For patients later in disease progression with high antibody titers, dilutions could be performed to adjust the concentration into the linear range of the assay. Testing a sample at various dilutions would also allow the calculation of specific antibody titers, which is not possible from a single undiluted sample.

Each sample in the longitudinal study was tested in duplicate on different days and by a different user to characterize the reproducibility and robustness of the platform (FIG. 12B). A strong correlation was found for each marker, with a Pearson’s r correlation of 0.98, 0.98, and 0.96 for S1, RBD, and N, respectively. The high correlation between replicates further emphasizes the quantitative nature and reproducibility of the platform for profiling the immune response to SARS-CoV-2.

### Example 19

### Concordance With Neutralizing Antibody Titers

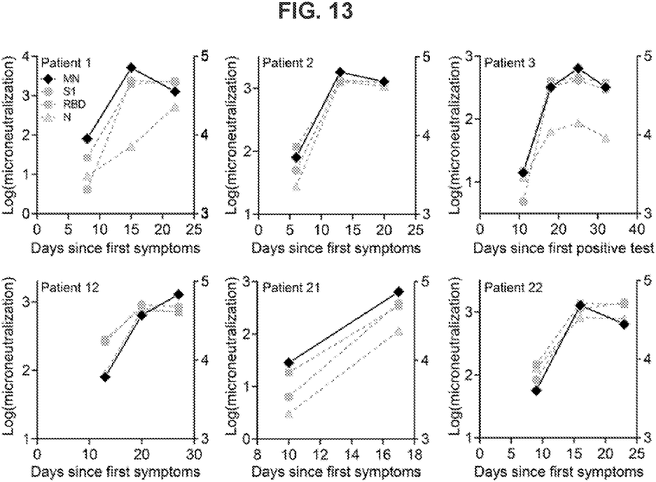

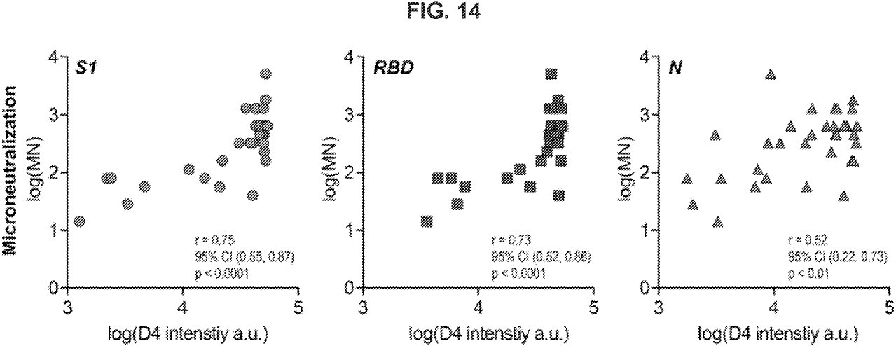

The performance of the DA-D4 assay was compared with a microneutralization assay that monitors functional neutralization of SARS-CoV-2 via neutralizing antibodies binding to the RBD. All six patients that were tracked longitudinally developed robust neutralizing antibodies, and the microneutralization titer was strongly concordant with DA-D4 assay readout for antibodies targeting S1 and the RBD of S1 (FIG. 13). Furthermore, a concordance analysis of the DA-D4 assay with the microneutralization assay for antibodies targeting S1 and RBD showed a strong correlation across 34 plasma samples tested (FIG. 14), as determined by a Pearson r> 0.70 (p < 0.0001). For antibodies targeting N, the concordance between the two assays was not as strong, with only a moderate correlation between the DA-D4 results and microneutralization data (FIG. 14). This is expected, as N resides inside the capsid of SARS-CoV-2 and is not relevant for functional neutralization. This is also reflected in the longitudinal sample set. For example, patient #1 at day 15 after symptom onset has strong neutralizing antibodies, as seen by the microneutralization assay, despite a weak overall antibody level for N. Although future studies are required to validate the ability of neutralizing antibodies to confer protection, these results suggest that the DA-D4 assay could be used as a supplement to live virus neutralization assays, which typically require > 48 hours and biosafety level 3 containment.

### Example 20

### Profiling Prognostic Biomarkers Concurrently With Serological Testing

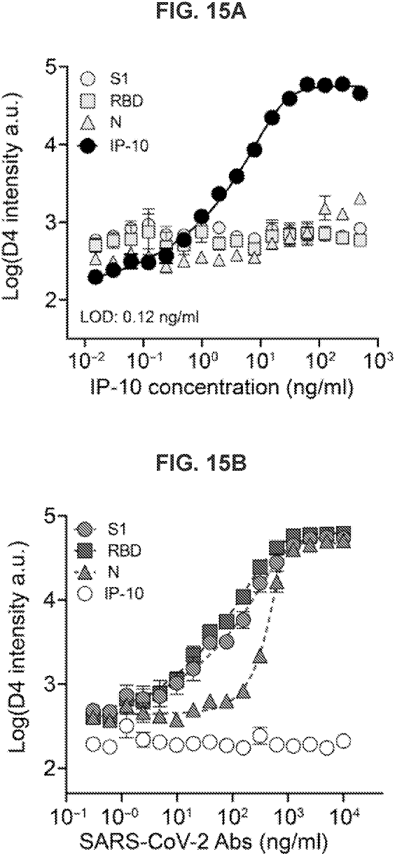

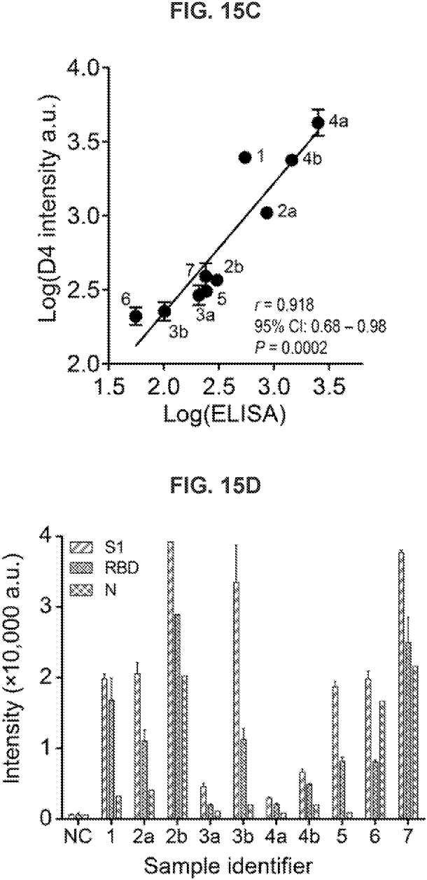

The feasibility of detecting a prognostic protein biomarker concurrently with serological profiling was investigated. This is motivated by the fact that others have identified potentially prognostic biomarkers that correlate well with disease severity and patient outcomes. Therefore, tracking antibody levels alongside prognostic biomarkers may provide clinically relevant information to inform interventions in the ICU for patients with a high probability of a poor outcome. As proof-of-concept, detection of IFN-γ-induced protein 10 (IP-10, CXCL10), a chemokine that recruits inflammatory cells to the site of inflammation and which has been shown to be elevated in severe disease and correlates with patient prognosis, was integrated into the DA-D4 assay using a traditional sandwich immunoassay approach. Prior to testing patient samples, the compatibility (i.e., non-cross reactivity) of the multiplexed serological assay with the IP-10 sandwich assay was evaluated. Open format chips were fabricated containing all necessary reagents for both COVID-19 serology and human IP-10 detection. First, a 15-point dilution series of recombinant human IP-10 was prepared and spiked into fetal bovine serum (FBS), spanning the relevant physiological range for COVID-19 patients identified elsewhere, and then samples were added to chips in triplicate in the absence of antibodies targeting SARS-CoV-2 antigens. A dose-dependent behavior for IP-10 response was observed with a low limit-of-detection of 0.12 ng/mL and minimal reactivity for SARS-CoV-2 capture antigens, confirming that the IP-10 assay components do not cross react with the serology components (FIG. 15A). Next, a dilution series of simulated seropositive samples was prepared and added to the open format chips. Across all concentrations of anti-SARS-CoV-2 antibodies, IP-10 capture antibody intensity was close to baseline, thus confirming that the serology components do not interfere with the IP-10 detection assay (FIG. 15B).

Having confirmed the compatibility of the IP-10 assay with multiplexed serology in the open D4 format, the performance of the assay was tested in patient samples. Ten COVID-19 positive plasma samples (from 7 patients) were procured from the ICU biorepository and were added undiluted to open format chips and then quantitatively assessed by the DA-D4. Separately, serum samples from the same patients were evaluated in parallel via LEGENDplex™ ELISA assay kits which report IP-10 concentration in pg/mL. A strong positive correlation was observed between the DA-D4 assay for IP-10 with ELISA across all 10 pairs of measurements, with a Pearson’s r of 0.918 (p = 0.0002, 95% Cl: 0.68 to 0.98) (FIG. 15C). Antibody reactivity towards S1, RBD, and N was also tested from the same samples and an additional sample of healthy pooled plasma (pre-COVID-19 negative control) (FIG. 15D). Although a strong relationship between antibody and IP-10 levels was not observed (data not shown), it was observed that in the patients for which multiple samples were tested, IP-10 decreased over time, while the levels of antibodies increased.

Overall, these results clearly show that the D4 assay format can simultaneously detect antibody response to foreign native SARS-CoV-2 antigens and a native protein biomarker from undiluted patient plasma. One of the benefits of detecting anti-SARS-CoV-2 antibodies from undiluted samples is that the sensitivity of the protein detection assay is not reduced because of dilution, allowing for the detection of chemokines and cytokines, which are present at very low concentrations even during disease state, directly from complex biological milieu. Detection of additional prognostic biomarkers could also be implemented on the same chip, as long as there is no cross-reactivity between the assay reagents for serology and prognosis. For example, a multiplexed assay has recently been developed to detect interleukin-6 (IL-6), N-terminal (NT)-pro-B-type natriuretic peptide (NT-proBNP), and D-dimer, which have all been implicated in disease progression and severity and could be added to the existing chip (FIGS. 16A-C). A recent study found that the ratio of IL-6 to IL-10 can be used to guide clinical decision making, which could be measured in the next generation of this assay.

As the COVID-19 pandemic unfolded, countries around the globe grappled with developing streamlined systems for diagnosis of acute infection using nucleic acid detection methods. Although there remains an urgent need for rapid and sensitive point-of-care tests for acute diagnosis, developing accurate and reliable serological assays has been deemed an equally important endeavor to complement existing diagnostic strategies. The challenge with developing an easy-to-use serology assay that can be broadly disseminated but that performs as well as centralized laboratory-based methods is highlighted by the large number of ELISA and LFA tests that have been developed. While LFAs are portable and easy-to-use and ELISAs are quantitative and highly sensitive, there remains a need for a technology that can merge the best attributes of each format.

The DA-D4 POCT is a promising platform to supplement existing diagnostic technologies to manage the COVID-19 pandemic because it marries the best attributes of LFAs and ELISAs: it is quantitative, easy to use, widely deployable, requires only a single 60 µL drop of blood, and can be performed with minimal user intervention. The SARS-CoV-2 DA-D4 assay can be used to measure antibody kinetics and seroconversion at the individual patient level directly from unprocessed blood or plasma. This test is highly sensitive and specific and is potentially suited for epidemiological surveillance at the population-level using low cost microfluidic cassettes that can be transported and stored for an extended period of time without a cold chain, and that require minimal user intervention to carry out the assay, which provide a quantitative readout using a low cost, hand-held detector.

A strong correlation between the DA-D4 assay readout (for S1 and the RBD of S1) and neutralizing antibody titers is shown, suggesting that this test may be useful in understanding efficacy and durability of natural or vaccine-induced humoral immunity, and to potentially inform disease prognosis and population-level immunity. An additional prognostic biomarker is also demonstrated to be easily incorporated into the test, which may be useful for monitoring disease severity and predict clinical outcomes. Combined, these attributes suggest this platform may also be useful on the individual patient level to aid in clinical decision making. While the results presented here mainly highlight the performance of the microfluidic chip, the open format architecture with up to 24 individual assays per glass slide may be useful for scenarios where higher throughput testing is demanded. The open format still has advantages compared to traditional ELISA because the open format only requires a single incubation step and one wash step, which reduces the hands-on time and equipment complexity required to complete the assay.

The DA-D4 has additional features that synergize to deliver a highly desirable serological assay. First, the double-antigen sandwich format (i.e., antibody bridging) has advantages over other serological assay formats. Because total antibody is detected rather than a single antibody isotype or subclass, seroconversion in patients can be detected earlier, which reduces the chances of a false negative result due to a test being administered too early in disease. Furthermore, because the labeled reagent does not have species specificity, the single assay kit could be used in pre-clinical vaccine development studies to measure antibody responses in experimental animals. The lack of species-specific detection antibodies also reduces the risk of high background signal caused by non-specific antibodies binding to the surface and subsequently being labeled.

Second, all reagents needed to complete the assay are incorporated onto the non-fouling POEGMA brush, which eliminates virtually all non-specific protein adsorption and cellular adhesion, thereby enabling an extremely low LOD directly from undiluted samples. Although many serological assays often dilute samples, the ability to test undiluted samples is advantageous, especially when combined with prognostic biomarker testing where dilution of low concentration analytes can lead to an undetectable signal. Testing multiple dilutions can still be performed using this test when antibody levels become high, which could be used to calculate specific titers. POEGMA also acts as a stabilizing substrate for printed reagents, enabling long term storage of chips without a cold chain. In this study, results were generated over the course of three months from the same batch of tests stored in silica desiccated pouches at room temperature and ambient humidity.

Third, this platform can be easily multiplexed, which can be used to capture a more detailed picture of the host immune response to SARS-CoV-2 infection by quantifying the antibody level induced to multiple viral antigens-in this case N, S1, and S1-RBD-from a single sample without sacrificing ease-of-use. This is because each viral antigen is deposited at a spatially discrete location, which allows for a single fluorescent tag to be used during fluorescence imaging of the chip, thereby simplifying assay readout compared to other multiplexing technologies such as Simoa or Luminex assays which rely on multiple different reporter molecules and a more complex readout. This method also allows simultaneous measurements of the concentration of potential prognostic biomarkers directly from plasma without compromising the performance of the multiplexed serological assay. There are currently no tests on the market that can probe for antibodies against multiple viral antigens and prognostic protein biomarkers simultaneously.

Fourth, this platform is designed for point-of-care deployment because it requires a single drop of blood that is readily obtained from a fingerstick. This droplet is injected into the sample port of a gravity driven microfluidic chip that requires no further user intervention beyond the concurrent addition of a few drops of wash buffer into a separate port. The assay runs by itself under the action of gravity and capillary action until all the fluid is drained from the microfluidic path by the absorbent pad at the bottom of the cassette, which fully absorbs and contains all liquid. The microfluidic chip relies only upon capillary action and gravity to drive fluid flow, which eliminates the need for pumps, valves, or actuators, and reduces the complexity and cost of the assay. This enables the assay to be read out at the point of sample collection using the D4Scope-a highly sensitive and inexpensive handheld detector developed to work with the microfluidic chip. The D4Scope images a chip and provides a quantitative readout in less than 5 seconds, does not require an external power source or laboratory infrastructure, and can wirelessly transmit the results to a remote server over Wi-Fi. While smartphone-based diagnostics are becoming more popular, a benefit of this platform is that it does not rely on smartphone hardware and software, which change rapidly. Combined, these attributes make this platform ideal for providing ELISA-like sensitivity and quantitation with the ease-of-use and scalability of LFAs.

The disclosed point of care assay for COVID-19 serology and prognosis would likely be most useful for serial quantification of antibody response and prognostic biomarkers to monitor symptomatic and severe cases where use of available therapeutics, such as antiviral or monoclonal therapies, is indicated. Further, it could be used to screen for patients with poor antibody responses who may benefit from convalescent plasma or monoclonal antibody therapy. This platform has potential utility in point-of-care settings such as ICUs, urgent care clinics, and at the point-of-use-at locations where periodic surveillance of healthcare workers and other essential workers in close proximity to others for extended periods of time such as assembly-line manufacturing or food processing plants- is desirable to assist in tracking clusters of disease and epidemiological studies. This platform could also be used as an inexpensive tool to study the longitudinal dynamics of antibody levels to inform re-infection potential, as coronavirus immunity often lasts only ~6 months 50. Similarly, it could be used to monitor vaccine-induced humoral immunity, which could help determine if boosters are needed in certain vaccinated individuals. This technology is suitable for low-resource settings across the globe, where eliminating the need for sample storage and transport to a centralized testing facility, and the attendant cold chain, is desirable, and where access to expensive, high-throughput clinical analyzers that process large volumes of serology and other sandwich immunoassays is limited. Similarly, remote and austere settings -such as the field-forward position of the military or other remote locations where pandemics often emerge- can also benefit from this platform, as the testing is carried out with a disposable cassette and a low-cost, light-weight, and handheld detector whose production can easily be scaled up to enable wide-spread and dispersed deployment.

While the results presented here are promising, there are several issues identified during this study that require further investigation prior to its deployment. First, the cohort of individuals with SARS-CoV-2 infection consisted of adults with clinically significant disease, which is not representative of the entire spectrum of COVID-19 disease severity. These samples were chosen to demonstrate proof-of-concept of the DA-D4 assay and because these samples were locally available through an existing biobank. A larger sample size that spans the disease severity spectrum is required to develop a more robust measure of sensitivity and specificity of the DA-D4 serology test for SARS-CoV-2. Similarly, demographics were not able to be matched in the negative control group, which may have introduced confounding variables in the analyses.

Furthermore, several of the samples that were tested saturated the readout of the assay, which limits the dynamics that can be measured once high antibody titers are achieved. This limitation could be addressed by testing individual samples on separate microfluidic chips at various dilutions, which would effectively increase the dynamic range of the assay and yield more precise quantitative titer. Additionally, because of the double antigen design of the assay, discrimination between specific antibody subclasses or isotypes is not possible, which has been shown to be important for other diseases. Despite these limitations, this assay is well poised to complement existing diagnostic solutions once additional validation studies encompassing larger patient cohorts are completed.

In summary, a COVID-19 serological assay has been developed that merges the benefits of LFAs and ELISAs. This test was used to simultaneously measure the antibody levels for multiple viral antigens and a potential prognostic biomarker directly from plasma and whole blood. For COVID-19 management, this platform may be useful to better understand patient antibody responses, provide actionable intelligence to physicians to guide interventions for hospitalized patients at the point-of-care, assess vaccine efficacy, and perform epidemiological studies. Furthermore, this platform is broadly applicable to other diseases where sensitive and quantitative antibody and or protein detection is desirable in settings without access to a centralized laboratory. Overall, this platform is a promising approach to democratize access to laboratory quality tests, by enabling rapid and decentralized testing with minimal user intervention to locations outside the hospital.

### Example 21

### Detection of Ovalbumin Protein in a Variety of Samples Using the Microfluidic Cassette

First, the concentration amount of printed detection reagent (ovalbumin detection antibody (dAb)) was optimized to maximize performance on the microfluidic cassette. A titration of three conditions was tested as 8-point dose response curves (0.1-100 ng/mL + 0 ng/mL blank, n = 2, in FBS, ~30-minute incubation). 36, 48, and 60 ng of total dAb at concentrations of 0.12, 0.16, and 0.20 mg/mL were used to print the desired amount of dAb. For this study, the slides were only imaged on the Genepix scanner, and not the D4Scope.

Evaluation of the responses from the ovalbumin dAb titration is shown in FIG. 17. Adding more dAb resulted in an increase in background fluorescence, while reducing dAb resulted in an overall attenuation in fluorescence response, both of which resulted in a decrease in sensitivity leading to the selection of 48 ng of total dAb for the microfluidic cassette.

Once the optimal printing conditions were identified, a larger scale experiment was conducted with those conditions to identify the performance metrics of the ovalbumin assay on the microfluidic D4 cassette in FBS. A 10-point dose response curve (0.01-100 ng/mL + 0 ng/mL blank, n = 4, FBS, ~30-minute incubation, 60 µL sample) was conducted using 40 individual microfluidic chips and was imaged on both the D4Scope and Genepix.

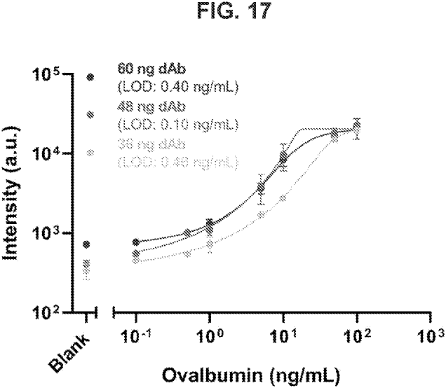

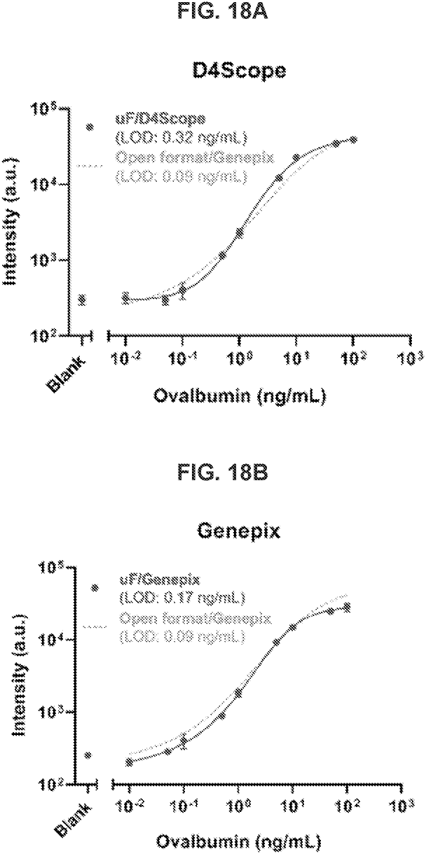

FIGS. 18A-B show the resulting FBS curves. The performance of the microfluidic cassette compared to the open format was consistent with what was previously observed in other applications where there is roughly a doubling in LOD from the open format to the microfluidic cassette when imaged on the Genepix, and another doubling when translating to the D4Scope. In this case, an increase from 0.09 ng/mL to 0.32 ng/mL was observed for the ovalbumin assay. Even with the expected and modest reduction in performance, the test still has a strong ~6 pM sensitivity. The consistency of the dose points in quadruplicates indicates the test is also capable of being highly quantitative. These curves will be used as a point of comparison for subsequent studies.

### Example 22

### Spike and Recovery Experiment

The spike and recovery experiment (0.01-300 ng/mL + 0 ng/mL blank, FBS, 30-minute incubation) was conducted with two operators. Operator #1 prepared and randomized the tested doses creating a key that would be required to know the ovalbumin concentration in each aliquot. Operator #2 took those randomized doses and ran them, analyzed the resulting images, and predicted the dose of each randomized aliquot based on the FBS dose response curve (FIGS. 18A-B) before being unblinded to correlate the predicted versus actual dose concentration.

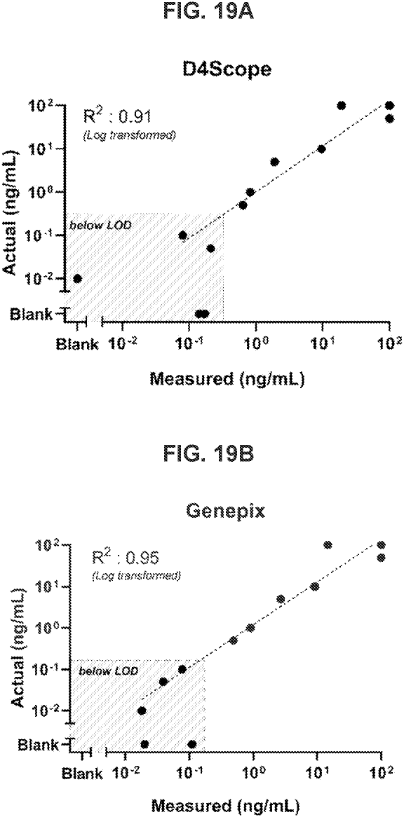

FIGS. 19A-B show the results from the spike and recovery experiment. In both cases, there was a strong correlation between the measured and actual dose concentration, especially in the dynamic range above the LOD for each condition. Most critically, in both cases the cassette successfully identified 100% of doses as being below or above the LOD. This is an important validation of the microfluidic cassette in conjunction with the portable D4Scope being able to distinguish dose concentrations from an unknown sample with sensitivities and specificities near the calculated LOD from analytical dose response curves.

Similar to the study conducted with the open format, it is important to characterize the microfluidic D4 cassette’s resilience to fluctuations in temperature. It is important to repeat this study in the microfluidic cassette architecture as there may be some cassette-specific influences such as more rapid residence time in the case of the elevated temperature environment.

All tested conditions were run as 8-point dose response curves (0.01-100 ng/mL + 0 ng/mL blank, 0, 0.1, 0.33, 2 ng/mL (n = 3) 0.01, 0.033, 10, 100 ng/mL (n = 2), in FBS, 30-minute incubation). A temperature and humidity probe were used to confirm all tested conditions. A room temperature (23° C.) and humidity (50%) condition was run as a comparative benchmark for the study. A 5° C. study was conducted in a refrigerator inside of a sealed bag with an amount of added silica desiccant to maintain the desired 55% ± 5% control humidity (as refrigerators are typically more humid than ambient humidity). A 40° C. study was conducted in an oven inside of a bag with small section of wet towel to maintain the desired 55% ± 5% control humidity (as the oven is close to 0% humidity once pre-heated). A 9% humidity experiment was conducted on the bench with an excess of silica desiccant to maintain the desired low humidity. An 80% humidity experiment was conducted on the bench with an excess of water saturated towels to maintain the desired high humidity. To prevent the cassettes from spending too much of their residence time in ambient conditions, cassettes were loaded with sample in sets of 3, before being added to their respective environmental conditions. This resulted in no more than 1-minute of time before the sample was loaded and the cassette was in the desired test condition.

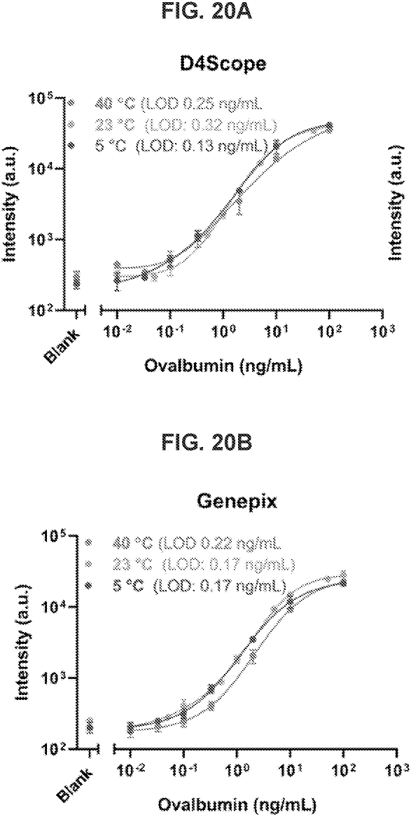

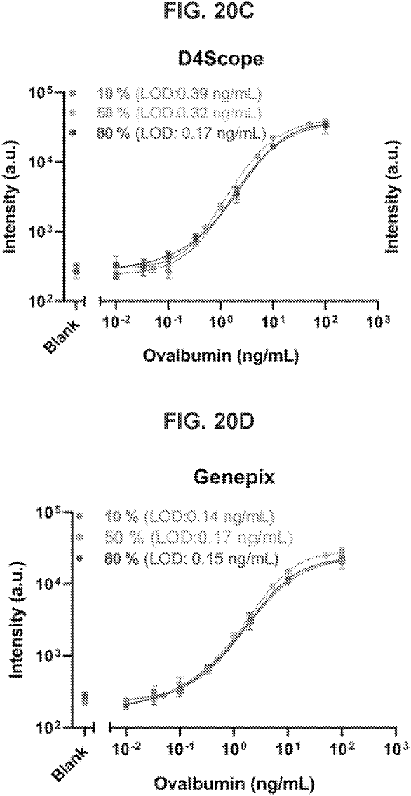

From a performance perspective, the microfluidic cassette proved resilient to extremes in temperature and humidity both when imaged on the Genepix and the D4Scope (FIGS. 20A-D). For temperature, this is somewhat at odds with what was observed in the open format test. If anything, the performance improves with reduced temperature rather than increased as you would expect as the antibody kinetics improve. It is important to note that the residence time increased by 3-5 minutes for the 5° C. condition, while it decreased by 3-5 minutes for the 40° C. This occurs because temperature has a direct impact on the viscosity of the sample and, as a result, the capillary flow rate. It is hypothesized that the improved kinetics as a function of increasing temperature is somewhat balanced out by the decreased residence time as a function of increasing temperature, resulting in more consistent results across varied temperatures. There was no noticeable change in performance as a function of humidity. This is to be expected since the microfluidic cassette is completely enclosed and should be insulated from external changes in relative humidity.

### Example 23

### Urine Study

All tested conditions were run as 8-point dose response curves (0.01-100 ng/mL + 0 ng/mL blank, 0, 0.01, 0.033, 1 ng/mL (n = 3) 10, 100 ng/mL (n = 2), in pooled human urine, 30-minute incubation). The samples were run identically to previously described dose response curves.

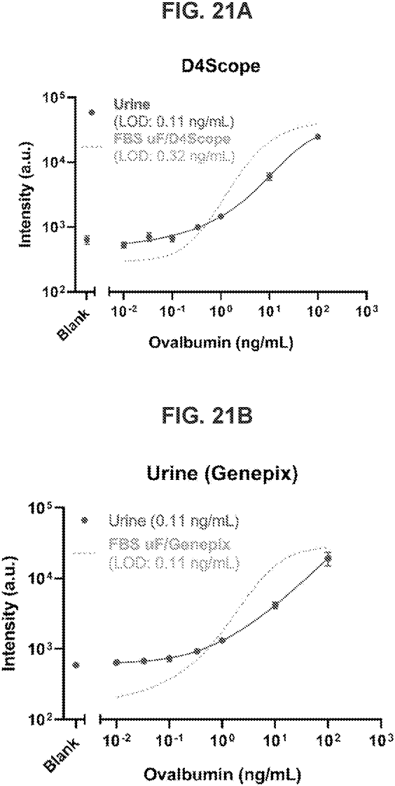

The pooled human urine dose response curve (FIGS. 21A-B) had a noticeably elevated background fluorescence and attenuation of the overall fluorescence in the dynamic range. This could be due to the relatively increased acidity, reduced protein content, or increased salt concentration when compared to serum. The gentle nature of the wash step in the cassette compared to the open format likely exasperates the increased background, since that did not manifest in the open format urine data. The change in curve shape did not result in reduced performance, as the calculated LOD (0.11 ng/mL) was comparable to the best sensitivities observed on the platform. This highlights that the cassette is fully capable of running urine samples, but that a universal dose response curve cannot be used to assess analyte concentration from any sample type. Rather, unique curves will be required specific to the sample type. The increased background and attenuated signal also indicate there is likely room for improved sensitivity with further sample-specific optimization.

### Example 24

### Blood Study

All tested conditions were run as 8-point dose response curves (0.01-100 ng/mL + 0 ng/mL blank, 0, 0.01, 0.033, 1 ng/mL (n = 3) 10, 100 ng/mL (n = 2), single donor human whole blood, 30-minute incubation). Single donor human whole blood was purchased commercially (Innovative Research). To test changes in performance as a function of hematocrit, two conditions were created: 30% and 65% hematocrit. These conditions were made by spinning the human blood at 2,000 rcf for 15 min in a 4° C. swinging bucket centrifuge. A crude estimation of hematocrit can be calculated by evaluating the relative volume occupied by the red blood cell layer and the total volume of the sample. Plasma was removed from the supernatant of some aliquots and then added to others to create the desired span of hematocrit percentages. The modified blood version of the microfluidic cassette was used for this study and has been previously described.

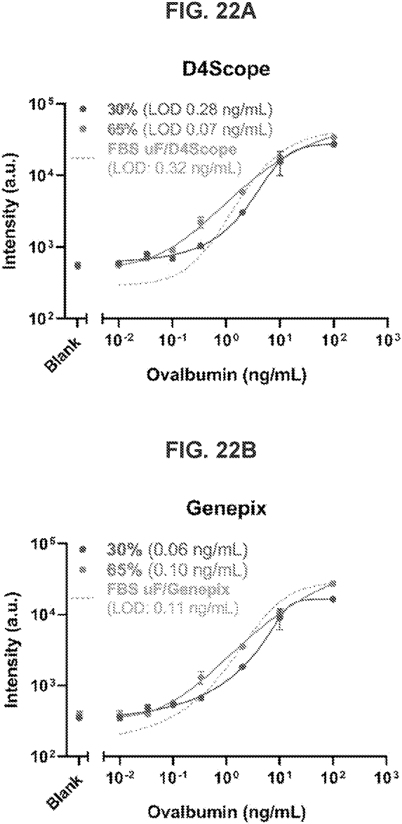

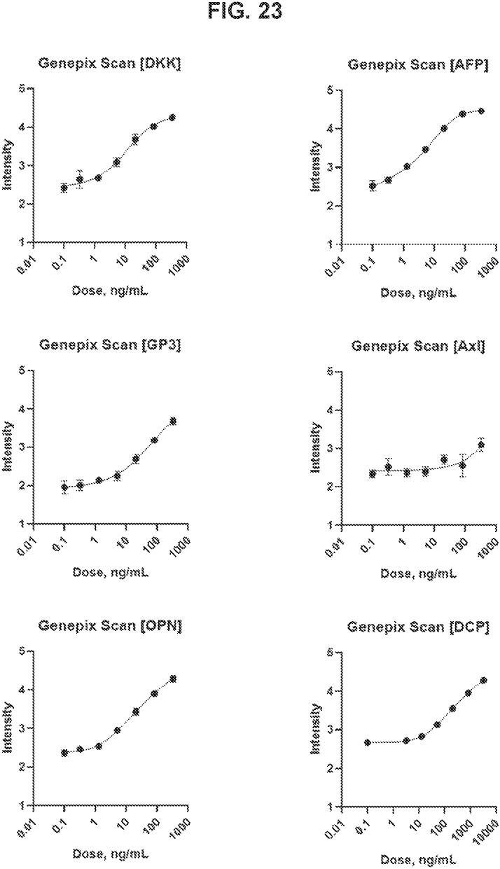

The two tested hematocrits span the physiologically relevant range and encompass conditions such as anemia and severe dehydration. FIGS. 22A-B show the results. There was a noticeable attenuation in fluorescence for the 30% hematocrit conditions when compared to the 65% condition. This was not evident in the same comparison on the open format dose response curve. There are a few potential causes to this shift. First, the higher hematocrit sample will have a higher viscosity, which directly impacts capillary flow rate and thus resulted in a ~4-5-minute increase in overall residence time. Second, the presence of anti-ovalbumin antibodies in the sample could be attenuating the dose response at lower hematocrits. The method of hematocrit modulation would result in a relative increase in anti-ovalbumin (if present) in the lower hematocrit solution. If this was the case, it would be expected that the same behavior would be observed in the open format. There is an increase in LOD as a function of decreasing hematocrit, but in the case of the dose response curves, there isn’t the same attenuation in the dynamic range and fluorescence like what is seen for the cassette. This could be because the relative amount of anti-ovalbumin from the donor for the open format test was less than that of the microfluidic. More likely, the attenuation was due to multiple factors, similar to what was observed in the temperature study. However, the results successfully highlight the ability for the cassette to test in undiluted whole blood of varied hematocrit with high sensitivity. The influence of hematocrit will need to be explored more deeply, but it makes sense to conduct these studies with a biomarker of clinical significance to eliminate the confounding factor of anti-ovalbumin in the sample. Table 4 provides a summary for how the microfluidic platform compares to the open format, and further how performance changes when imaged on the D4Scope versus Genepix scanner.

TABLE 4

Summary of how the microfluidic platform compares to the open format and how performance changes when imaged on the D4Scope versus Genepix scanner

| | Open format | Microfluidic cassette | Microfluidic cassette |
|---|---|---|---|
| | Genepix | Genepix | D4Scope |
| Test conditions | LOD (ng/mL) | LOD (ng/mL) | LOD (ng/mL) |
| FBS (RT / 50% RH) | 0.09 | 0.17 | 0.32 |
| FBS (RT / 10% RH) | 0.10 | 0.14 | 0.39 |
| FBS (RT / 80% RH) | 0.22 | 0.15 | 0.17 |
| FBS (40° C. / 50% RH) | 0.04 | 0.22 | 0.25 |
| FBS (5° C. / 50% RH) | 0.15 | 0.17 | 0.13 |
| Pooled Human Urine | 0.17 | 0.11 | 0.11 |
| Whole blood (30% hematocrit) | 0.55 | 0.07 | 0.28 |
| Whole blood (65% hematocrit) | 0.13 | 0.06 | 0.10 |

### Example 25

### Microfluidic Cassette With Hepatocellular Carcinoma (HCC) Panel

A hepatocellular carcinoma (HCC) panel was introduced onto the microfluidic cassette. It contains 6 prospective markers for HCC. FIG. 23 shows the panel being run in spiked FBS and analyzed on the Genepix scanner (n = 4). This demonstrates that the microfluidic cassette can be multiplexed for up to at least six biomarkers, and potentially more, with good performance.

### Example 26

### Alternative Microfluidic Cassette Designs and Functionality

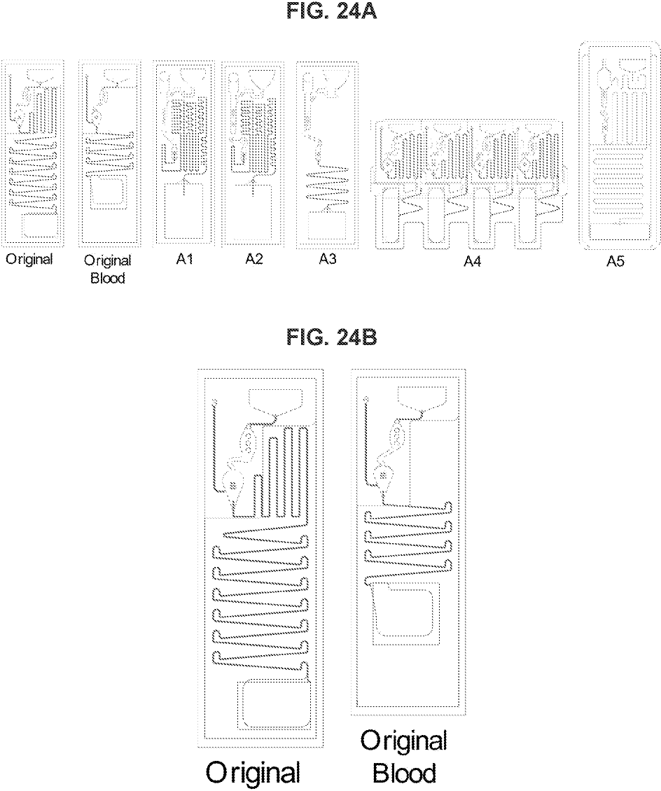

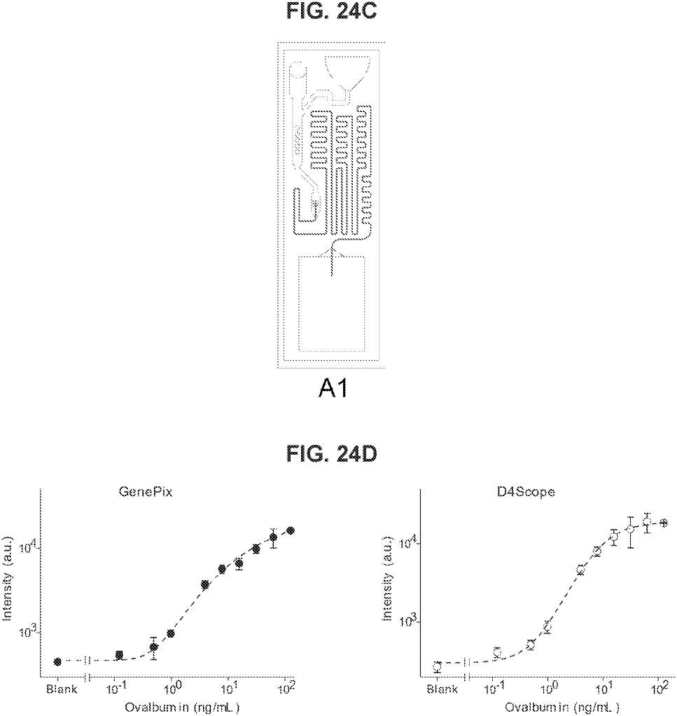

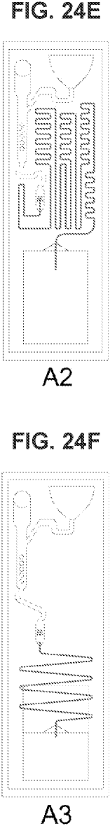

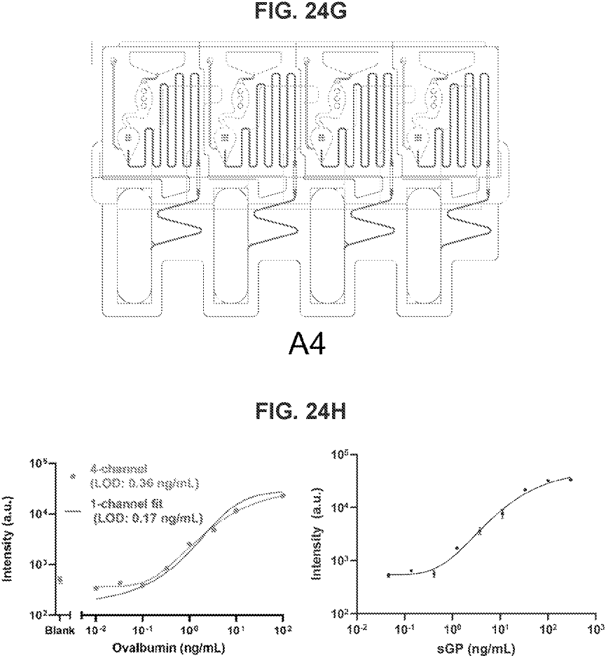

The “original” and “original blood” cassette designs are illustrated in FIGS. 24A-B. Various alternative microfluidic cassette versions have been developed from the original designs. Alternative cassette designs A1-A5, illustrated in FIG. 24A and FIGS. 24C-I, demonstrate examples of how the geometry and orientation of the chambers, channels, and inlets can be modified while the core functionality remains intact. Each design has a reaction chamber where the detection and capture reagents are printed, and each design has a timing channel. This reaction chamber always has more height than the timing channels. Timing channels are 0.157 mm, while reaction chambers are 1 mm in height, but these relative size differences can be modified in future iterations. All designs have a lower portion of the reaction chamber where capture is printed and a higher portion of the reaction chamber where detection is printed. There is an offset between these sections to prevent streaking of labelled reagent into the capture lower region, and a mixer to better dissolve the detection into the sample before entering the lower portion of the reaction chamber. The exact geometries of the offset and mixer change for each design. The length, width, and presence of vertical loops are also changeable based on the assay type, sample type, and desired residence time. All designs have a wash reservoir where wash buffer is added immediately after the sample is added. All designs have an outlet with an absorbent pad. The direction and exact geometry of the timing channel/outlet interface changes by design. While all designs have a sample inlet, some designs have a small sample inlet connected to a small channel that delivers sample into the bottom of the reaction chamber. These inlets have a backflow reduction valve and must be loaded with a pipette tip. Other designs have a large opening that drops the sample into the top of the reaction chamber, where the sample can be loaded in with a dropper. Each design version of the cassette is fabricated in an identical way to the original version but has a different footprint for each layer. The individual characteristics and differences for each alternative design are described below.

Design A1 (FIG. 24C): Designed to operate using a drop of sample rather than requiring the user to introduce the sample with a micropipette. The reaction chamber is modified to have a large sample inlet situated at the top of the chamber that is large enough to be able to add the sample with a dropper. This is different from the original version that has a smaller inlet that feeds into the lower portion of the reaction chamber, requiring a pipette tip to fill. Furthermore, the chamber of A1 is always vented to atmospheric pressure, unlike the original version, which results in the wash buffer freely filling the reaction chamber once added rather than the wash buffer entering the reaction chamber at a rate equal to the rate of sample leaving the chamber through the timing channels. The sample runs through the top of the reaction chamber and then settles near the bottom where the detection and capture reagents are located. This allows for a wider range of volumes to be utilized since the volume that the reaction chamber occupies is less than the typical 60-70 µL of sample. Because of the longer reaction chamber, the hydrostatic pressure component of fluid flow is more dominant, resulting in the need for more vertical loops in the timing channel. This version of the cassette has also been validated in an ovalbumin model system, as shown in FIG. 24D.

Design A2 (FIG. 24E): Designed to conduct a competition assay rather than a standard sandwich assay. In this design, the detection reagent is introduced as a direct competitor to the protein of interest in the sample, both trying to bind to the same capture spot. Through experimentation, it was discovered that it is critical for the detection and sample to mix well before interacting with the capture spots. The reason for this is that binding could be biased depending on if more analyte-rich sample or more detection-rich sample reaches the capture spot first (from poor mixing). This is counter to a standard sandwich immunoassay, where the order of binding events is less important. In this system, intensity is inversely related to protein concentration. The reaction chamber of this version introduces a more aggressive offset-mixer channel prior to the capture spot region to better promote sample/detection homogeneity before incubation with the capture spots. A2 is designed to run competition assays in non-whole blood samples including, but not limited to, FBS, serum, buffered saline, and urine.

Design A3 (FIG. 24F): This design is the same as A2 but modified to conduct a competition assay format with undiluted human whole blood. Notable changes to the design include an angled slope in the offset-mixer to prevent sedimentation and clogging of red blood cells during the wash stage of operation, and there are no vertical loops in the timing channel (similar rationale to the blood version of the original cassette design).

Design A4 (FIG. 24G): This design is an experimental version of the original design used to increase the density of tests per POEGMA substrate. The significant POEGMA real estate consumed by non-reaction chamber features of previous microfluidic designs was the motivator for this design. Knowing that incubation can be driven primarily through the size and length of the vertical loops, design A4 was created to fit four full microfluidic cassettes onto a single POEGMA-coated glass slide. Since the POEGMA substrate is the most expensive component of the fabrication, this drastically decreases the cost per assay. This version adds a bottom acrylic layer that is on the same plane as the POEGMA substrate. Timing is controlled primarily through the vertical timing loops. By extending the vertical timing loops to be taller, it reduces the overall channel length required and reduces the footprint for a single test, eliminating the need for lengthy downstream timing channels. The downstream channels and absorbent waste collection pad are housed away from the POEGMA onto an acrylic-backed substrate, which is a unique feature to versions A4 and A5 of the cassettes. To accomplish this, a bridging feature was introduced in this design. This is a uniquely designed “bridge” feature that allows the sample to cross the POEGMA-acrylic border on the bottom substrate at the edge of the POEGMA slide without leaking. The channel height at the intersection was briefly increased to 1 mm by cutting the channel into the middle acrylic layer, rather than the bottom adhesive layer where all other channels are housed. In this same area, the bottom adhesive was left uncut, creating an adhesive seal over the POEGMA-acrylic intersection without leaking. This allowed seamless transition from a POEGMA substrate to an acrylic substrate. Acrylic-on-acrylic channels had previously been attempted, but the high contact angle of the acrylic made it difficult to design reliable timing channels. However, with the acrylic-on-acrylic channels being downstream from the reaction chamber, hydrostatic pressure dominates over the poor capillary flow and provides enough pressure to drive the sample to the absorbent pad and initiate washing and drying. This design has been demonstrated in both an ovalbumin model as well as for the detection of sGP (for Ebola diagnostics), as shown in FIG. 24H. Other than these modifications listed, the reaction chamber, inlets, outlets, and core functionality remain unchanged compared to the original version.

Design A5 (FIG. 24I): This design is an experimental version of the cassette that houses all timing channels away from the POEGMA substrate brush (acrylic-on-acrylic channels). This version combines the features of A2 and A4 and only uses half of a POEGMA slide. The microfluidic cassette has more width to create a frame on the back of the assembly that the POEGMA piece can be easily inserted into. Similar to design A4, this version adds a bottom acrylic layer that is on the same plane as the POEGMA substrate. This cassette design was the first to successfully demonstrate all acrylic-on-acrylic timing channels. This was accomplished because of the difference in reaction chamber design from the original version of the cassette to version A4. Primarily, the open nature of the sample inlet in A2 allows the wash buffer to freely flow into the reaction chamber, increasing the hydrostatic pressure acting on the fluid front downstream. This increased pressure, combined with the long and narrow geometry of the reaction chamber, allowed for vertical loops to be designed that create reliable timing channels, which were previously unable to be achieved. Instead of the same “bridge” design seen in A4, this design features a “tunnel.” The timing channel traverses a very small channel on the POEGMA substrate before it reaches a circular tunnel feature built into the adhesive layers and middle acrylic. This routes the sample and timing channel above the POEGMA onto a timing channel designed in the top adhesive layer between the middle and top acrylic layers. This cassette features a narrower offset-mixer upstream from the capture region to decrease the POEGMA footprint. This design also features a small wash buffer delay channel, which was implemented because it sometimes takes a few seconds for the sample to settle down to the bottom of the reaction chamber after being added. The small delay allows the user to add wash buffer immediately after the sample without any worry of the rare event of premature mixing occurring into the sample.

What is claimed:

1. A multi-layered microfluidic assay device comprising a cassette comprising a plurality of layers comprising:
a substrate layer comprising a non-fouling polymer layer coated on a glass substrate; and assay reagents disposed upon the substrate layer;
a microfluidic layer comprising a channel and a reaction area in fluid communication with each other and to inlets and outlets, the ultimate microfluidic layer adjacent to a cover layer.

2. The device of claim 1, wherein the microfluidic layer comprises:
a channel layer comprising a continuous circuitous channel in fluid communication with a reaction layer; and
a reaction layer comprising cutouts for a reaction chamber, an inlet, and an outlet, each in fluid communication with the channel layer, the reaction layer being sandwiched between the channel layer on one side and the cover layer on the other side.

3. The device of claim 2, wherein the reaction chamber comprises an upper reaction chamber, a lower reaction chamber, and an offset mixing channel fluidly connecting the upper and lower reaction chambers.

4. The device of claim 3, wherein the offset mixing channel comprises a P-trap bend to prevent clogging.

5. The device of claim 3, wherein the assay reagents comprise one or more detection reagents and one or more capture reagents that are spatially separated and disposed on the non-fouling polymer layer to align with the lower and upper reaction chambers.

6. The device of claim 5, wherein the detection reagents are placed on layers of trehalose, disposed on the substrate spatially at a position corresponding to the upper reaction chamber; and of the capture reagents are disposed on the substrate spatially at a position corresponding to the lower reaction chamber.

7. The device of claim 2, wherein the cover layer is attached to the reaction layer via an adhesive layer.

8. The device of claim 2, wherein the channel layer, the reaction layer, and the cover layer are interconnectedly vented to ambient atmospheric pressure.

9. The device of claim 2, wherein the cover layer comprises one or more inlets and outlets in fluid communication with the microfluidic layers and with a reservoir at the inlets and an absorbent waste pad at the outlets.

10. The device of claim 1, wherein the non-fouling polymer layer is a poly(oligoethylene glycol methyl ether methacrylate) (POEGMA).

11. The device of claim 2, wherein the channel layer is an adhesive layer or an injection molded plastic layer.

12. The device of claim 2, wherein the reaction layer is acrylic.

13. The device of claim 2, wherein the continuous circuitous channel has longitudinal/vertical and lateral/horizontal orientations.

14. The device of claim 1, wherein the device is configured to operate in a substantially vertical orientation aligned with gravity.

15. The device of claim 1, wherein the device is configured to operate via gravity-assisted capillary flow for sample transit through the microfluidic layer.

16. The device of claim 2, wherein the reaction layer has a thickness of 0.2 to 3.0 mm.

17. The device of claim 1, wherein the reaction chamber length ranges from about 10 mm to about 40 mm.

18. The device of claim 1, wherein the reaction chamber width ranges from about 2 mm to about 5 mm.

19. The device of claim 2, wherein the total channel length ranges from about 50 mm to about 600 mm.

20. The device of claim 2, wherein the channel width ranges from about 0.05 mm to about 2 mm.

21. The device of claim 1, wherein the channel thickness ranges from about 0.05 mm to about 0.5 mm.

22. The device of claim 1, wherein the residence time of a sample in the chamber ranges from about 5 minutes to about 2 hours.

23. The device of claim 1, wherein the residence time of a sample in the channel after it has emptied from the reaction chamber ranges from about 5 minutes to about 1 hours.

24. The device of claim 2, wherein the channel comprises from about 1 to 8 horizontal/lateral loops and 0 to 8 and vertical/longitudinal loops.

25. The device of claim 1, wherein the channel comprises one or a plurality of vertical/longitudinal oriented loops.

26. The device of claim 1, wherein the channel does not comprise any vertical/longitudinal oriented loops.

27. The device of claim 1, wherein the sample inlet delivers the sample directly into the lower reaction chamber.

28. The device of claim 27, wherein the sample inlet has a diameter from about 0.5 mm to about 1.5 mm.

29. The device of claim 1, wherein the sample inlet delivers the sample directly into the upper reaction chamber.

30. The device of claim 29, wherein the sample inlet has a diameter from about 1.0 mm to about 5.0 mm.

31. The device of claim 1, further comprising an acrylic substrate layer on the same plane as the POEGMA substrate which creates a POEGMA-acrylic border.

32. The device of claim 31, further comprising a bridge feature that allows the sample to cross the POEGMA-acrylic border on the bottom acrylic substrate layer at the edge of the POEGMA substrate without leaking.

33. The device of claim 31, further comprising a tunnel feature built into the adhesive layers and the bottom acrylic substrate layer.

34. The device of claim 31, further comprising a wash buffer delay channel.

35. The device of claim 1, wherein the device has functional improvements as compared to conventional devices, including: a longer shelf life; extended incubation times; room temperature storage and operation; low sample volume required for testing; capability of detecting multiple biomarkers simultaneously; and capability of being configured for multiple assay types with minor modifications to design.

36. Use of the device of claim 1 for analyzing a biological sample by measuring a concentration level of an analyte in the biological sample.

37. A method for analyzing a biological sample by measuring a concentration level of an analyte, the method comprising:
(a) orienting the device of claim 1 with gravity with the sample inlet at the top;
(b) loading a sample into the sample inlet;
(c) loading a wash buffer into the wash reservoir of the device;
(d) allowing the sample and wash buffer to enter and traverse completely through the device;
(e) imaging the device to measure a signal for the target analytes and controls; and
(f) determining the concentration of the analyte.

38. The method of claim 37, wherein the sample comprises whole blood, serum, plasma, urine, tears, sweat, saliva, lymph, cerebrospinal fluid, fecal extract, cellular or tissue extracts, or any other aqueous sample.

39. The method of claim 37, wherein the analyte is a biomolecule from an infectious agent, cancer, or is a biomarker for cardiovascular disease, or metabolic disorder.

40. The method of claim 37, wherein the analyte is a biomolecule or biomarker from a host response to an infectious agent, cancer, cardiovascular disease, or metabolic disorder.

41. The method of claim 37, wherein the analyte is a biomolecule or biomarker for SARS-CoV-2 or Ebola.

42. The method of claim 37, wherein the analyte is a cancer biomolecule or biomarker.

43. The method of claim 37, wherein the analyte is a biomolecule or biomarker associated with hepatocellular carcinoma.

44. A method for fabricating a microfluidic cassette assay device comprising a substrate layer having microfluidic geometry and a reagent layer disposed adjacent to the substrate layer, the method comprising:
(a) depositing a poly(oligoethylene glycol methyl ether methacrylate) (POEGMA) layer onto a glass substrate;
(b) depositing a trehalose layer upon the POEGMA layer;
(c) depositing a detection reagent onto the trehalose layers and a capture reagent onto the POEGMA layer at sites corresponding to a reaction chamber;
(d) adhering complementary layers of acrylic and adhesive sheets having microfluidic geometries onto the POEGMA substrate coated glass slide base, wherein the microfluidic geometries comprise a sample inlet, a wash reservoir, a reaction chamber comprising an upper chamber and a lower chamber separated by an offset mixing channel, a circuitous channel comprising a plurality of loops, and an outlet; and
(e) attaching a wash reservoir and absorbent waste pad.

45. The method of claim 44, wherein the acrylic and adhesive sheets are laser-cut to form microfluidic geometries prior to adhering onto the POEGMA substrate.

46. The method of claim 44, wherein the acrylic and adhesive sheets are injection molded to form microfluidic geometries prior to adhering onto the POEGMA substrate.

47. The method of claim 44, wherein the capture reagent and detection reagent are inkjet-printed and spatially separated to align with the corresponding microfluidic geometry of the substrate layer.

48. The method of claim 44, wherein the capture reagent is printed in the bottom region of the reaction chamber and the detection reagent is printed in the top region of the reaction chamber.

49. A microfluidic assay system comprising:
the microfluidic assay device of claim 1;
a stand;
a wash buffer; and
a sample applicator.

50. A kit comprising:
the microfluidic assay device of claim 1;
a stand;
a wash buffer; and
a sample applicator.

51. A multi-layered microfluidic assay device comprising a cassette comprising a plurality of layers comprising:
a substrate layer comprising a non-fouling polymer layer coated on a glass substrate; and assay reagents disposed upon the substrate layer;
a microfluidic layer comprising a channel layer and a reaction layer in fluid communication with each other and to inlets and outlets, the ultimate microfluidic layer adjacent to a cover layer;
the channel layer comprising a continuous circuitous channel in fluid communication with the reaction layer;
the reaction layer comprising cutouts for a reaction chamber, an inlet, and an outlet, each in fluid communication with the channel layer, the reaction layer being sandwiched between the channel layer on one side and the cover layer on an opposing side; and
the reaction chamber comprising an upper reaction chamber, a lower reaction chamber, and an offset mixing channel fluidly connecting the upper and lower reaction chambers.

52. Use of the device of claim 51 for analyzing a biological sample by measuring a concentration level of an analyte in the biological sample.
